(19) United States
(12) Patent Application Publication
(10) Pub. No.: US 2009/0252647 A1
(43) Pub. Date: Oct. 8, 2009

(54) COMPOSITIONS AND METHODS FOR APPLYING ANTIMICROBIALS TO SUBSTRATES

(75) Inventor: Richard Allen Orofino, Northport, NY (US)

Correspondence Address: KALOW & SPRINGUT LLP, 488 MADISON AVENUE, 19TH FLOOR, NEW YORK, NY, 10022, US

(73) Assignee: Crosstex International, Inc., Hauppauge, NY (US)

(21) Appl. No.: 12/061,128

(22) Filed: Apr. 2, 2008

(51) Int. Cl. A61L 2/18

(52) U.S. Cl. 422/28

(57) ABSTRACT

Methods and compositions are provided that control spread of pathogens and prevent infections. In various embodiments, a liquid impervious substrate having an antimicrobial disposed thereon is provided. In one embodiment, a method of antimicrobially treating a substrate is provided, comprising contacting the substrate with an effective amount of a water stable composition, comprising i) water; ii) a product that is formed by reacting in water: a) an organosilane of the formula Rₙ SiX₄₋ₙ where n is an integer of from 0 to 3; each R is, independently, a nonhydrolyzable organic group; and each X is, independently, a hydrolyzable group, with b) a polyol containing at least three hydroxy groups, wherein all of the hydroxy groups are separated by at least three intervening atoms, wherein the polyol is not hydroxyethyl cellulose, wherein the product is a monomer, oligomer, or a combination thereof, and iii) a wetting agent in an amount of 0.01% w/w or w/v to 10% w/w or w/v based on the weight of the total composition for a period of time sufficient to antimicrobially treat the substrate. In various embodiments, the substrate is a face mask or dental product.

### BACKGROUND

Recently, there has been great interest in different ways to reduce the risk of infection not only in nursing homes, hospitals and hospices throughout the nation, but also in the doctor's and dentist's office, as well as in non-healthcare settings such as businesses, offices, schools and other places where people congregate. The healthcare and non-healthcare environments contain a diverse population of microorganisms, which can cause infection. Microorganisms (e.g., bacteria, fungi, yeast, molds and viruses) in air and water, on surfaces, on skin, in bodily fluid (e.g., blood, saliva, secretions, wound exudate, etc.), and other sources tend to be the biggest players in the spread of infection. Not only are patients at risk of developing infection, but also are the visitors, nurses, doctors, or other healthcare and non-healthcare workers that come into contact with these infectious sources.

Medical knowledge and public awareness of ways in which infections are transmitted is helping to reduce spread of infections. Infection prevention and control procedures involving universal precautions such as hand washing, wearing gloves, gowns, masks and other protective equipment and covering open wounds has also helped reduced the spread of infections. However, exposure to pathogens such as HIV (human immunodeficiency virus), hepatitis B virus (HBV), hepatitis C virus (HCV), tuberculosis, Avian influenza, Staphylococcus aureus, vancomycin-resistant enterococci (VRE), Escherichia coli, Klebsiella pneumoniae, Pseudomonas aeruginosa, and other pathogens can still be problematic and pose risks to patients, visitors, and healthcare and non-healthcare workers. Exposure in some cases leads to the actual infectious disease, and in some severe cases, death.

Even with universal precautions in place, exposures to infectious diseases still occur. For example, there are a small number of instances when HIV has been acquired through contact with non-intact skin or mucous membranes. Research suggests that the risk of HIV infection after mucous membrane exposure (e.g., splashes of infected blood in the eye or mouth) is less than 1 in 1000. Nevertheless, this is still understandably an area of considerable concern for many healthcare and non-health workers, particularly where there are large quantities of blood or bodily fluid exposure (e.g., cardiac surgery, dental procedures, etc.).

Tuberculosis (TB) infection is another pathogen that is an occupational risk to the heath care worker. TB is spread from person to person through the air and usually attacks the lungs, but can attack almost any part of the body. It is estimated that 50-80% of hospital employees working for 15 years or more have been exposed to tuberculosis.

Conventional methods and compositions for reducing transmission of infection have been fairly effective. However, there is a need for new methods and compositions to effectively control spread of pathogens and prevent infections in the healthcare and non-healthcare settings. Methods and compositions that inhibit, prevent or control infections are still needed.

### SUMMARY

Methods and compositions are provided that control spread of pathogens and prevent infections. In various embodiments, the antimicrobial compositions and methods kill 99% or more of the microorganisms that they come in contact with. In various embodiments, a liquid impervious substrate having an antimicrobial disposed thereon is provided. The antimicrobial is applied to the substrate with a wetting agent that allows the antimicrobial to disperse and adhere to parts or the entire surface of the substrate. In various embodiments, an antimicrobial face mask is provided that kills microorganisms that come in contact with the face mask in healthcare and non-healthcare settings.

In one embodiment, a method of treating a substrate is provided, comprising contacting the substrate with a composition comprising an effective amount of an organosilane and a wetting agent for a period of time sufficient to antimicrobially treat the substrate.

In another embodiment, a method of antimicrobially treating a substrate is provided, comprising contacting the substrate with an effective amount of a water stable composition, comprising i) water; ii) a product that is formed by reacting in water: a) an organosilane of the formula Rn SiX4−n where n is an integer of from 0 to 3; each R is, independently, a nonhydrolizable organic group; and each X is, independently, a hydrolyzable group, with b) a polyol containing at least three hydroxy groups, wherein all of the hydroxy groups are separated by at least three intervening atoms, wherein the polyol is not hydroxyethyl cellulose, wherein the product is a monomer, oligomer, or a combination thereof, and iii) a wetting agent in an amount of 0.01% w/w or w/v to 10% w/w or w/v based on the weight of the total composition for a period of time sufficient to antimicrobially treat the substrate.

In yet another embodiment, a method of antimicrobially coating a substrate is provided, comprising contacting the substrate with an effective amount of a water stable composition, comprising i) water; ii) a product that is formed by reacting in water: a) an organosilane of the formula Rn SiX4−n where n is an integer of from 0 to 3; each R is, independently, a nonhydrolizable organic group; and each X is, independently, a hydrolyzable group, with b) a polyol containing at least three hydroxy groups, wherein all of the hydroxy groups are separated by at least three intervening atoms, wherein the polyol is not hydroxyethyl cellulose, wherein the product is a monomer, oligomer, or a combination thereof, and iii) a wetting agent in an amount of 0.01% w/w or w/v to 10% w/w or w/v based on the weight of the total composition for a period of time sufficient to antimicrobially coat the substrate.

In an exemplary embodiment, a method of antimicrobially enhancing a substrate is provided, comprising contacting the substrate with an effective amount of a water stable composition, comprising i) water; ii) a product that is formed by reacting in water: a) an organosilane of the formula Rn SiX4−n where n is an integer of from 0 to 3; each R is, independently, a nonhydrolizable organic group; and each X is, independently, a hydrolyzable group, with b) a polyol containing at least three hydroxy groups, wherein all of the hydroxy groups are separated by at least three intervening atoms, wherein the polyol is not hydroxyethyl cellulose, wherein the product is a monomer, oligomer, or a combination thereof, and iii) a wetting agent in an amount of 0.01% w/w or w/v to 10% w/w or w/v based on the weight of the total composition for a period of time sufficient to antimicrobially enhance the substrate.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE DRAWINGS

FIG. 1 illustrates a typical substrate that the antimicrobial composition is applied to in the form of a face mask. In this illustration a face mask for medical, dental, surgical, and non-healthcare use is provided. An antimicrobial amount of the organosilane is applied to the outer surface or layer of the mask that faces the patient or outside environment.

FIG. 2 illustrates a view of a substrate, in this case, non-woven polymer strands having the antimicrobial composition coated on the surface of the polymer. In this illustrated embodiment, some of the polymer surface has an antimicrobial amount of the organosilane applied to it. It will be understood that some, all, or substantially all of the polymer surface will have the antimicrobial applied to it.

FIG. 3 illustrates a polymer surface having the antimicrobial organosilane applied to the surface, where the organosilane associates with the surface of the substrate through, for example, ionic bonding, covalent bonding, hydrogen bonding, electrostatic interaction, hydrophobic, hydrophilic or other interaction with the surface of the substrate. The antimicrobial activity in this illustration is exerted when the microorganism usually having a negative charge is attracted to the positively charged nitrogen, when the microorganism comes into contact with the carbon chain, the microbe is killed or the growth of the microbe is inhibited.

FIG. 4 shows a typical reaction scheme for preparing 3-(trimethoxysilyl)propyl-dimethyloctadecyl ammonium chloride, and subsequent hydrolysis and conversion to the fully-complexed pentaerythritol-stabilized form.

FIG. 5 illustrates an embodiment of a perspective view of a face mask that has an absorbent material located on the inner surface of the mask.

FIG. 6 illustrates an embodiment of a perspective view of the face mask of FIG. 4 shown on a user.

FIG. 7 illustrates an embodiment of a back view of the face mask of FIG. 4.

FIG. 8 illustrates an embodiment of a cutaway view of the body portion showing the mask as being made of a plurality of layers.

FIG. 9 illustrates an embodiment of the face mask sections tested that had organosilane disposed on sections of the mask.

FIG. 10 illustrates an embodiment of the face mask sections tested that have proper distribution of the organosilane disposed on it and results on MRSA bacterial colonies applied to the mask.

It is to be understood that the figures are not drawn to scale. Further, the relation between objects in a figure may not be to scale, and may in fact have a reverse relationship as to size. The figures are intended to bring understanding and clarity to the structure of each object shown, and thus, some features may be exaggerated in order to illustrate a specific feature of a structure.

### DETAILED DESCRIPTION

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

It is noted that, as used in this specification and the appended claims, the singular forms “a,” “an,” and “the,” include plural referents unless expressly and unequivocally limited to one referent. Thus, for example, reference to “a wetting agent ” includes one, two or more wetting agents.

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents, which may be included within the invention as defined by the appended claims.

Unless stated otherwise, the following terms and phrases as used herein are intended to include the following meanings:

The term “alkyl” as used herein refers to a branched or unbranched saturated hydrocarbon group of 1 to 24 carbon atoms, such as methyl (“Me”), ethyl (“Et”), n-propyl, isopropyl, n-butyl, isobutyl, t-butyl, octyl, decyl, tetradecyl, hexadecyl, eicosyl, tetracosyl or the like. In various embodiments, alkyl groups herein contain from 1 to 12 carbon atoms. The term “lower alkyl” intends an alkyl group of from one to six carbon atoms or one to four carbon atoms. The term “cycloalkyl” intends a cyclic alkyl group of from three to eight or five or six carbon atoms. “Lower alkyl alcohol” refers to lower alkyl having attached thereto one or more hydroxy moieties, such as, but not limited to, —CH₂CH₂OH, CH₂CH(OH)CH₃, CH₂OH, CH₂CH₂CH₂OH, CH₂CH₂CH(OH)CH₃, CH₂CH₂CH(OH)CH₂OH, or CH₂CH(OH)CH(OH)CH₃.

The term “alkoxy” as used herein intends an alkyl group bound through a single, terminal ether linkage; that is, an “alkoxy” group may be defined as —OR where R is alkyl as defined above. A “lower alkoxy” group intends an alkoxy group containing from one to six or one to four, carbon atoms. “Polyether” refers to a compound or moiety possessing multiple ether linkages, such as, but not limited to, polyethylene glycols or polypropylene glycols. “Polyalkylethers” refers to alkyls interconnected by or otherwise possessing multiple ether linkages.

As used herein, “optional” or “optionally” means that the subsequently described event or circumstance may or may not occur, and that the description includes instances where said event or circumstance occurs and instances where it does not. For example, the phrase “optionally substituted lower alkyl” means that the lower alkyl group may or may not be substituted and that the description includes both unsubstituted lower alkyl and lower alkyl where there is substitution.

As used herein, the term “antimicrobial” is used in its general sense to refer to the property of the described compound, product, composition or article to prevent or reduce the growth, spread, formation or other livelihood of organisms such as bacteria, viruses, protozoa, molds, fungi (including algae), or other organisms. The term antimicrobial includes chemicals that kill (deactivate) or slow the growth of microbes. The antimicrobial may have antibacterial agents (which kill bacteria), antiviral agents (which kill viruses), antifungal agents (which kill fungi), and antiparasitic drugs (which kill parasites). In various embodiments, the antimicrobial comprises an organosilane.

By the term “effective amount” of a compound, product, or composition as provided herein, it is meant a sufficient amount of the compound, product or composition to provide the desired result. In various embodiments, the effective amount is an antimicrobial amount, which refers to the use of the compounds, products, or compositions wherein the organosilane has antimicrobial activity, along with other ingredients, surfactants, fillers, wetting agents, pigments, dyes, antimigrants, etc., to create a composition or solution capable of fulfilling its original purpose, based upon the other ingredients, and also of providing antimicrobial protection during the particular application.

In various embodiments, the effective amount includes additional antimicrobial activity to such compositions or solutions where no such activity previously existed, or to the increase of antimicrobial activity wherein the starting compositions or solutions inherently possessed antimicrobial activity. In various embodiments, the effective amount inhibits or kills microbes (e.g., bacteria, viruses, fungi, yeast, protozoa, parasites, and/or molds).

The term “aryl” as used herein refers to a compound or moiety whose molecules have a ring or multiple ring structure characteristic of benzene, naphthalene, phenanthrene, anthracene, etc., e.g., either the six-carbon ring of benzene or the condensed six-carbon rings of the other aromatic derivatives, including, but not limited to phenyl, benzyl, naphthyl, benzylidine, xylil, styrene, styryl, phenethyl, phenylene, benzenetriyl, etc. As used herein, the term “aromatic” refers to the group of unsaturated cyclic hydrocarbons, typified by benzene, having a 6-carbon ring containing three double bonds or multiple attached benzene rings. Moreover, certain five membered cyclic compounds, such as furan(heterocyclic), are analogous to aromatic compounds. Aromatics include the cyclic compounds based upon a benzene functionality, as specified for “aryl” above. Moreover, the term “cyclic” is used to refer to all aliphatic or aromatic hydrocarbons having one or more closed rings, whether unsaturated or saturated. In various embodiments, cyclic compounds possess rings of from 5 to 7 carbon atoms or 6 carbon atoms. Such rings fall into three classes: alicyclic, aromatic (“arene”), and heterocyclic. Moreover, when used with respect to cyclic compounds or moieties, the term “unsaturated” refers to such compound or moiety possessing at least one double or triple bond or otherwise constituting an aromatic compound or moiety. Moreover, the term “saturated” refers to compounds or moieties possessing no double or triple bonds, e.g., where all available valence bonds of an atom, especially carbon, are attached to other atoms.

The term “heteroaryl” refers to an aryl where one or more of the carbon atoms of a ring have been substituted with a heteroatom, including, but not limited to, O, N, or S. Similarly, the term “heterocyclic” refers to a cyclic compound or moiety where one or more of the carbon atoms of the ring has been substituted with a heteroatom, including, but not limited to, O, N, or S.

As used herein, especially in reference to alkyl and alkoxy, the term “lower” refers to a moiety having from 1 to 6 carbon atoms, or 1 to 4 carbon atoms.

As used herein, the term “suitable” is used to refer to a moiety, which is compatible with the compounds, products, or compositions as provided herein for the stated purpose. Suitability for the stated purpose may be determined by one of ordinary skill in the art using only routine experimentation.

As used herein, “substituted” is used to refer, generally, to a carbon or suitable heteroatom having a hydrogen or other atom removed and replaced with a further moiety. In one embodiment, halogen, hydroxy, and nitrogen based substitutions of hydrocarbon hydrogens are contemplated as within the scope of the present invention for the claimed structures. Moreover, it is intended that “substituted” refer to substitutions, which do not change the basic and novel utility of the underlying compounds, products or compositions of the present invention. “Unsubstituted” refers to a structure wherein the reference atom does not have any further moieties attached thereto or substituted therefor.

As used herein, “branched” is used to refer, generally, to a moiety having a carbon chain backbone, e.g., alkyl or alkoxy, wherein the backbone may contain one or more subordinate carbon chain branches. For example, isobutyl, t-butyl, isopropyl, CH₂CH₂C(CH₃)(H)CH₂CH₃, CH₂C(CH₂CH₃)(H)CH2CH₃, CH₂CH₂C(CH₃)₂CH₃, and CH₂CH₂C(CH₃)₃ would all be considered branched moieties. Moreover, it is intended that “branched” variations of the moieties herein described refer to variations, which do not change the basic and novel utility of the underlying compounds, products or compositions of the present invention. “Unbranched” refers to a structure wherein the carbon chain does not have any branches thereon, i.e., where the carbon chain extends in a direct line.

As used herein, the term “acyl” refers to organic acid derived moieties of the formula RCOX where R is an organic molecule and X, instead of being hydroxy, is replaced with another substituent, preferably, a suitable anion, such as a halogen including, but not limited to, F, Cl, Br or I.

As used herein, the term “perfluoro” or “perfluoro-analog” refers to a hydrocarbon where the hydrogen atoms attached to carbons have been replaced with F atoms. Preferably, but not necessarily, in perfluoro-analogs, most if not all of the H atoms are replaced with F atoms. A “fluoro-” analog is contemplated to indicate a hydrocarbon where at least one hydrogen atom attached to a carbon is replaced with an F atom.

As used herein, the term “stabilizer” is used to refer to the class of polyols as specified herein wherein any two of the at least three hydroxy groups are separated by at least three atoms. Such compounds have been found to stabilize organosilanes by preventing self-condensation or other inactivation of the resulting compounds and products.

The term “halogen” is used to refer to Fluorine “F”, Chlorine “Cl”, Bromine “Br”, Iodine “I”, and Astatine “At”. In various embodiments, halogen or halide refers to F, Cl, or Br. The term “halide” is meant to include these halogens.

As used herein, “substrate” refers to any article, product, or other surface that can be treated with the organosilane compositions. Suitable substrates are generally characterized as preferably having a negatively charged surface of oxygen atoms, or any surface capable of electrostatically, ionically, covalently or by other interaction adhering or binding to the organosilane. In various embodiments, the adhering or binding occurs at the silicon atom of the organosilane portion of the compounds, products, or compositions but such binding is not a requirement. Therefore, as used herein, the term “adhere” or “associate” is meant to refer to ionic, covalent, electrostatic, or other chemical attachment of a compound, product or composition to a substrate. Substrate refers to any suitable substrate which is or may come into contact with medical patients (human or animal), medical caregivers, bodily fluids, or any other source of contamination or infection generally associated with hospitals, clinics, physician's offices, etc. Substrates, include, but are not limited to, surgical, medical, or dental face masks or other types of masks.

As used herein, “hydrolyzable” refers to whether the moiety is capable of or prone to hydrolysis (i.e., splitting of the molecule or moiety into two or more new molecules or moieties) in aqueous or other suitable media. Conversely, “nonhydrolizable” refers to moieties that are not prone to or capable of hydrolysis in aqueous or other suitable media.

As used herein, “cationic” is used to refer to any compound, ion or moiety possessing a positive charge. Moreover, “anionic” is used to refer to any compound, ion or moiety possessing a negative charge. Furthermore, “monovalent” and “divalent” are used to refer to moieties having valances of one and two, respectively. Moreover, as used herein, the term “salt” is meant to apply in its generally defined sense as “compound formed by replacing all or part of the hydrogen ions of an acid with one or more cations of a base.” Suitable salts may be formed by replacing a hydrogen ion of a moiety with a cation, such as K⁺, Na⁺, Ca₂⁺, Mg₂⁺, etc.

As used herein, “wetting agent” includes any material that aids the ability of a liquid (e.g., hydrous liquid) to spread or “wet” a surface. Wetting agents include compounds that reduce surface tension of the liquid and/or substrate. Wetting agents can be anionic, cationic, non-ionic or amphoteric (zwiterionic) compounds or combinations thereof.

The headings below are not meant to limit the disclosure in any way; embodiments under any one heading may be used in conjunction with embodiments under any other heading.

### Antimicrobial Compositions

In various embodiments, a method of antimicrobially treating a substrate with a composition is provided. The composition comprising: i) water; ii) a product that is formed by reacting in water: a) an organosilane of the formula Rn SiX4−n where n is an integer of from 0 to 3; each R is, independently, a nonhydrolizable organic group; and each X is, independently, a hydrolyzable group, with b) a polyol containing at least three hydroxy groups, wherein all of the hydroxy groups are separated by at least three intervening atoms, wherein the polyol is not hydroxyethyl cellulose, wherein the product is a monomer, oligomer, or a combination thereof, and iii) a wetting agent in an amount of 0.01% w/w or w/v to 10% w/w or w/v based on the weight of the total composition. The substrate is treated with the composition for a period of time sufficient to antimicrobially treat the substrate.

Any of the water stabilized organosilane compositions described in U.S. Pat. Nos. 5,959,014, 6,221,944 and 6,632,805 assigned to Emory University are suitable for use. The entire disclosures of U.S. Pat. Nos. 5,959,014, 6,221,944 and 6,632,805 are herein incorporated by reference into the present application.

In particular, a wide variety of organosilanes are preferred and comprise the general formula Rₙ SiX₄₋ₙ where n is an integer of from 0 to 3, preferably 0 to 2; R is a nonhydrolyzable organic group, such as but not limited to, alkyl, aromatic, organofunctional, or a combination thereof; and X is halogen, such as but not limited to, Cl, Br, or I, or X is hydroxy, alkoxy such as methoxy or ethoxy, acetoxy, or unsubstituted or substituted acyl. For such organosilanes, X is prone to react with various hydroxyl containing molecules to liberate methanol or ethanol. However, it is this reaction of X, which is responsible for the instability and, often, water-insolubility of such organosilanes.

In a further embodiment, about 0.001% to about 15% by weight (w/w or w/v) of an organosilane containing hydrolyzable groups and from about 0.25 to about 5.0 molar equivalents, or from 1 to about 2 molar equivalents of a polyol stabilizer are used and a wetting agent. The compounds, products and compositions are prepared by admixing or dissolving any of the described polyol stabilizers in less than the final desired volume of water, adding any of the desired organosilanes to the water solution, and then diluting further with water to the desired concentration and then adding the wetting agent before, during or after addition of the stabilizer and/or organosilane. This preparation method is preferably for water soluble stabilizers, such as pentaerythritol, tris(hydroxymethyl)ethane, tris(hydroxymethyl)nitromethane, etc.

In various embodiments, from about 0.5% to about 10% w/w or w/v of organosilane comprises an effective antimicrobial amount to apply or impregnate the substrate. In other embodiments, from about 1% to about 5% w/w or w/v of organosilane in water comprises an effective antimicrobial amount to apply or impregnate the substrate. In other embodiments, from 1.25% to about 2.5% w/w or w/v of organosilane in water comprises an effective antimicrobial amount to apply or impregnate the substrate.

Alternatively, where the stabilizers are not sufficiently water-soluble, additional stability is achieved by directly forming a trioxasilabicyclooctyl species and the organosilane may be reacted with the stabilizer in a non-aqueous solvent. In such an alternative preparation, the remaining solvent (e.g., methanol) is liberated via distillation. Both of these methods provide stable, clear solutions of the organosilane, which are capable of coating, spraying, and/or impregnating substrates with the organosilane upon treatment of the surface with the solution. The solutions are stable within a pH range of from about 2.0 to about 10.5, preferably from about 2.0 to about 7.0, for extended periods, up to several months or longer. Higher pH stability (>7.0) is also within the scope of the present invention.

Numerous art-known organosilanes are suitable for the present stabilization procedures to produce water-stabilized compounds, products and compositions. U.S. Pat. Nos. 5,411,585; 5,064,613; 5,145,592, and the publication entitled “A Guide to DC Silane Coupling Agent” (Dow Corning, 1990) disclose many suitable organosilanes. The contents of these references are incorporated in their entirety herein by reference.

In various embodiments, the organosilane comprises 3-(trimethoxysilyl)propyl-dimethyloctadecyl ammonium chloride, 3-(trimethoxysilyl)propylmethyldi(decyl)-ammonium chloride, 3-chloropropyltrimethylsilane, 3-chloropropyl-trimethoxysilane, octadecyltrimethoxysilane, or perfluorooctyltriethoxysilane and the polyol is pentaerythritol, dipentaerythritol, tripentaerythritol, tetrapentaerythritol, tris(hydroxymethyl)ethane, tris(hydroxymethyl)propane, tris(hydroxymethyl)-nitromethane, tris(hydroxymethyl)aminomethane, or tris(hydroxymethyl)methanetrimethyl ammonium iodide.

In various embodiments, the composition comprises a water-stable composition comprising water and a product that is formed by reacting in water a) an organosilane of the formula Rₙ SiX₄₋ₙ, where n is an integer of from 0 to 2; each R is, independently, a nonhydrolyzable organic group further wherein each R is, independently, alkyl, alkyl alcohol, or aromatic; and each X is, independently, a hydrolyzable group, further wherein each X is, independently, hydroxy, alkoxy, halogen, acetyl, acetoxy, acyl, a hydroxylated solid or liquid polymeric moiety, polyether or polyalkylether, with b) a polyol containing at least three hydroxy groups, wherein all of the hydroxy groups are separated by at least three intervening atoms, wherein the polyol is of the formula I:

{R₁₁(W)ₚ}(R₃₃)₃₋ₒC {(CH₂)_{q}OH}ₒ

wherein

o is an integer of from 2 to 3;

q is an integer of from 1 to 2;

p is an integer of from 0 to 1;

W is alkyl, polyether, aryl or heteroaryl;

R₁₁ and R₃₃ are, independently, halogen, H, CH₂OH, N(CH₂ CH₂OH)₂ CH₃⁺V⁻, NH₂, NO₂, N(H)(CH₂)₃OSO₃H, N⁺(CH₃)₂(CH₂)₃SO₃⁻, N(CH₃)₃⁺V⁻, (CH₂)OPO₃H₂, (CH₂)PO₃H₂,

N(H)R₃₄ (CF₂)ₑCF₃ where e is an integer of from 1 to about 22 and R₃₄ is CO or SO₂,

(W)ₚZO where Z is H, alkyl, aryl, or heteroaryl,

(W)ₚZS(O)ᵣ where Z is H, Na, a suitable mono- or divalent cation, alkyl, aryl, or heteroaryl and r is an integer from 0 to 2,

(W)ₚZ₁Z₂N where Z₁ and Z₂ are, independently, H, alkyl, aryl, or heteroaryl,

(W)ₚZ₃Z₄Z₅N⁺Q⁻ where Z₃, Z₄, and Z₅ are, independently, H, alkyl, aryl, or heteroaryl and where Q⁻¹ is a suitable anionic moiety to form a salt, or

(W)ₚZ₆PO₃ where Z₆ is H, Na, a suitable mono- or divalent cation, alkyl, aryl, or heteroaryl; and V⁻ is a suitable anionic moiety to form the salt of the compound of formula I; and a wetting agent in an amount of 0.01% w/w or w/v to 10% w/w or w/v based on the weight of the total composition.

In various embodiments, W is alkyl of from 1 to 22 carbon atoms. Examples of W include, but are not limited to, polyether, polypropyleneglycol, polyethyleneglycol, aryl, phenyl, benzyl, or heteroaryl, wherein the one or more heteroatoms are, independently, N, O, or S.

Some organosilanes suitable for use in the present invention include organosilicon quaternary ammonium salt compounds. Examples of these compounds include, but are not limited to, 3-(triethoxysilyl)-propyl-dimethyloctadecyl ammonium chloride, 3-(tri-methoxysilyl)propyl-methyl-dioctyl ammonium chloride, 3-(trimethoxysilyl)propyl-dimethyldecyl ammonium chloride, 3-(trimethoxysilyl)-propyl-methyldidecyl ammonium chloride, 3-(trimethoxy-silyl)propyl-dimethyldodecyl ammonium chloride, 3-(tri-methoxysilyl)-propyl-methyldidodecyl ammonium chloride, 3-(trimethoxy-silyl)propyl-dimethyltetradecyl ammonium chloride, 3-(trimethoxy-silyl)propyl-methyldihexadecyl ammonium chloride, 3-(trimethoxysilyl)propyl-dimethyloctadecyl ammonium chloride, octadecylaminodimethyltrimethoxysilylpropyl ammonium chloride, octadecyldimethyltrimethoxysilylpropyl ammonium chloride AEM 5700 (aegis) or a combination thereof.

Stabilizers suitable for use, in various embodiments, contain at least three hydroxy groups, where any two of the three hydroxy groups are preferably separated by at least three intervening atoms, i.e., (HO-A-B—C—OH). Such stabilizers can stabilize aqueous solutions of the above-described organosilanes Rₙ SiX₄₋ₙ and are generally useful for stabilization of all such solutions where n is an integer from 0 to 2 and where water solubility or minimization or prevention of water-induced, silanol self-condensation (and associated polymerization) is desired. In particular, preferred stabilizers are polyols containing three or more OH groups and having at least three carbon atoms separating any two OH groups.

In various embodiments, the polyol comprises pentaerythritol, dipentaerythritol, tripentaerythritol, tetrapentaerythritol, tris(hydroxymethyl)ethane, tris(hydroxymethyl)-propane, tris(hydroxymethyl)nitromethane, tris(hydroxymethyl)aminomethane, tris(hydroxymethyl)methane trimethyl ammonium iodide, or tetrakis(hydroxymethyl)-phosphonium chloride.

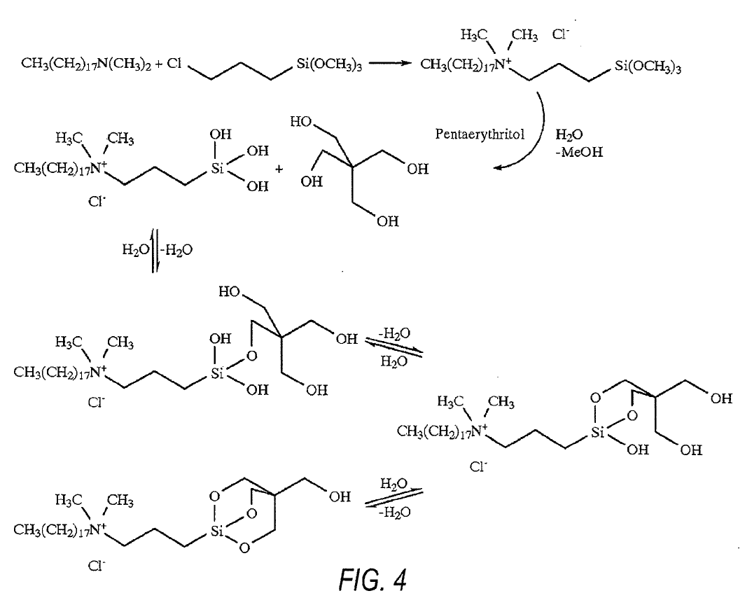

FIG. 4 shows a typical reaction scheme for preparing 3-(trimethoxysilyl)propyl-dimethyloctadecyl ammonium chloride, and subsequent hydrolysis and conversion to the fully-complexed pentaerythritol-stabilized form. FIG. 4 assumes initial water hydrolysis of the 3-(trimethoxysilyl)propyl-dimethyloctadecyl ammonium chloride, but it is understood that formulation of the stabilized form can also occur directly from the trimethoxy species by alcoholysis.

In various embodiments of the preferred formulation, the organosilane comprises 3-(trihydroxysilyl)propyldimethyloctadecyl ammonium chloride, the polyol comprises pentaerythritol, and the wetting agent comprises ethoxylated alcohol and glycol ethers. In various embodiments, from about 0.5% to about 10% w/w or w/v of organosilane comprises an effective antimicrobial amount to apply or impregnate the substrate. In other embodiments, from about 1% to about 5% w/w or w/v of organosilane in water comprises an effective antimicrobial amount to apply or impregnate the substrate. In other embodiments, from 1.25% to about 2.5% w/w or w/v of organosilane in water comprises an effective antimicrobial amount to apply or impregnate the substrate.

Wetting Agents

The antimicrobial composition optionally may comprise a wetting agent. Wetting agents have the property of modifying the characteristics of the contact between a liquid and the substrate surface to promote more rapid wetting of the substrate surface. In various embodiments, the wetting agent improve distribution of the antimicrobial on the surface of the substrate and bonding of the antimicrobial to the surface of the substrate.

Wetting agents can be anionic, cationic, non-ionic or amphoteric (zwiterionic) or combinations thereof. Typically, wetting agents are compounds comprising both hydrophilic and hydrophobic or lipophobic groups. In view of their dual hydrophilic and hydrophobic nature, wetting agents tend to concentrate at the interfaces of aqueous mixtures; the hydrophilic part of the wetting agent orients itself towards the aqueous phase and the hydrophobic parts orients itself away from the aqueous phase into the second phase. In various embodiments, the hydrophobic part of a wetting agent molecule may be derived from a hydrocarbon containing 8 to 20 carbon atoms (e.g. fatty acids, paraffins, olefins, alkylbenzenes). The hydrophilic portion may either ionize in aqueous solutions (cationic, anionic) or remain un-ionized (non-ionic). Wetting agents and wetting agent mixtures may also be amphoteric or zwitterionic.

Nonionic wetting agents are surface active agents, which do not dissociate into ions in aqueous solutions, unlike anionic surfactants which have a negative charge and cationic surfactants which have a positive charge in aqueous solution. Nonionic wetting agents show excellent solvency, low foam properties and chemical stability. It is thought that nonionic surfactants are mild on the skin even at high loadings and long-term exposure. The hydrophilic group of nonionic surfactants typically comprise a polymerized alkene oxide (water soluble polyether with 10 to 100 units length typically). They are prepared typically by polymerization of ethylene oxide, propylene oxide, and butylene oxide in the same molecule.

Wetting agents optionally may be present in the composition at up to 20 wt. % (w/w or w/v) and often up to 10 wt. %. In various embodiments, the wetting agent is present at a level of at least 0.001 wt. %, often at least 10 wt. %. Wetting agents may be present in any range of values inclusive of those stated above.

In various embodiments, the wetting agent is present in an amount of from about 5% to about 10% (w/w or w/v) and can improve microorganism kill approaching about 99% or even 100%.

In various embodiments, by adding the wetting agent, it improves the shelf life of the antimicrobial substrate (e.g., face mask). For example, when no wetting agent is used in some embodiments, the shelf life will be 0 days or I day (the antimicrobial will not attach to the substrate). In some embodiments, when the wetting agent is used the shelf life will increase to 6 months, 1 year, 2 years, 3 years, 4 years, 5 years or longer.

Examples of wetting agents include, but are not limited to, nonylphenol and its ethoxylates (e.g., ethoxylated nonylphenol, nonylphenol mono- and diethoylates, nonylphenoxy carboxylic acids, ethoxylated alcohol and glycol ethers, or combination thereof), alcohols (e.g., ethanol, cetyl alcohol, isopropyl alcohol), soap or a combination thereof. Wetting agents include anionic wetting agents, such as sodium lauryl sulfate; sodium lauryl ether sulfate, cationic stearalkonium chloride; benzalkonium chloride, quaternary ammonium compounds; amine compounds, and the like. Non-ionic wetting agents include coco diethanol-amide alcohol ethoxylates; linear primary alcohol polyethoxylate alkylphenol ethoxylates; alcohol ethoxylates; polyethylene glycol, esters of polyethylene glycol; fatty acid alkanolamides. Amphoteric wetting agents such as cocoamphocarboxyglycinate; cocamidopropylbetaine, and the like.

The wetting agent which is employed may facilitate coating, spraying, or dispensing the compositions. The wetting agent surprisingly improves the efficacy of the compositions. For example, in one embodiment, the wetting agent improves antimicrobial activity so there is 100% kill of the microbes (Example 2).

In various embodiments, the wetting agent includes ethoxylated alcohols and ethoxylated nonylphenols, which should have very low foaming properties. Foam formulation should be avoided during the production process as they can reduce the uniformity of the coating on the substrate. The concentration on wetting agent varies usually from 0.001% to 20% depending on the concentration, the morphology and the surface properties of the organosilane used.

### Substrates

In various embodiments, a method is provided for making an antimicrobial substrate. The method includes treating the substrate with an antimicrobial composition, wherein the composition includes contacting the substrate with an effective amount of a water stable composition, comprising i) water; ii) a product that is formed by reacting in water: a) an organosilane of the formula Rₙ SiX₄₋ₙ where n is an integer of from 0 to 3; each R is, independently, a nonhydrolyzable organic group; and each X is, independently, a hydrolyzable group, with b) a polyol containing at least three hydroxy groups, wherein all of the hydroxy groups are separated by at least three intervening atoms, wherein the polyol is not hydroxyethyl cellulose, wherein the product is a monomer, oligomer, or a combination thereof, and iii) a wetting agent in an amount of 0.01% w/w or w/v to 10% w/w or w/v based on the weight of the total composition for a period of time sufficient to antimicrobially treat the substrate.

Substrates include synthetic and natural fibers, filaments or yams. Suitable synthetic fibers, filaments, or yarns include, but are not limited to, polyolefins, polyesters, and polyamides such as nylons, polyurethanes, halogenated polyolefins, polyacrylates, polyureas, polyacrylonitriles, as well as copolymers and polymer blends. Suitable natural fibers include cotton, rayon, jute, hemp, and the like, which may be present as staple fibers or spun into yams.

The substrate may optionally be pretreated with a wetting agent. In various embodiments, the wetting agent and the organosilane are mixed to form a mixture and then the mixture is contacted with the substrate. Thus, the need for pretreatment of the substrate is avoided.

In various embodiments, the substrate may have no wetting agent applied to it. Instead, the substrate is corona treated before the organosilane is contacted with it.

Substrates also include various materials, including foam materials, nonwoven, woven, and knit fabrics or webs, fibrous materials, and sponges. The term “nonwoven web” or “nonwoven fabric” refers to a web or fabric having a structure of individual fibers that are interlaid in an irregular manner. In contrast, knit or woven fabrics have fibers that are interlaid in a regular manner.

In various embodiments, the substrate can be made by spunbound and melt blown processes. “Spunbound” refers to small diameter fibers that are “spun” by extruding molten thermoplastic material in the form of filaments from a plurality of fine, usually circular, capillaries of a spinneret, and then rapidly reducing the diameter of the extruded filaments. Typically, the filaments are bonded together by passage between the rolls of a heated calender. “Melt blown” refers to a process of extruding molten thermoplastic material through a plurality of fine, typically circular, die capillaries as molten threads or filaments into a high velocity, typically heated, gas stream (e.g., air), which reduces the diameter of the filaments and deposits the filaments on a collecting surface to form a web of randomly disbursed melt blown fibers.

Non-limiting examples of particular substrates include single and multi-layer nonwoven constructions, dressings, drapes, gowns, disposable diapers, liners, blood pressure cuff liners, gloves, filter media, air filters, face masks (e.g., surgical, medical, or industrial commercial dental masks), orthopedic cast padding/stockinettes, upholstery, clothing, sponges, tissue, plastics, containers, curtains, tents, backpacks, underwear, outerwear apparel, polyurethane and polyethylene foam, surgical caps, shoe covers, shoes, socks, towels, disposal wipes, hosiery and intimate apparel, plastics, adhesives, paper, or the like.

In various embodiments, the substrate may have string, rubber band, strap, belt, adhesive, clip, tape, zipper, tie, Velcro® snap, or the like disposed on the substrate to allow the substrate to be attached to the healthcare personnel, patient, or user.

A dental product includes an article used for dental procedures. Suitable dental products are disclosed in the Crosstex Dental Catalogue 2007/8, the entire disclosure is herein incorporated by reference. A dental product includes a pouch, a sterilized pouch, a saliva ejector, a high volume evacuator, a dental paper, an articulating paper, a latex dental dam, a non-latex dental dam, a tongue depressor, a tongue retractor, a dental tray, a dental fluoride tray, a cotton roll, a dental mixing pad, an amalgam mixing pad, a latex glove, a non-latex glove, a nitrile glove, a dental non-woven product, a dental woven product, a dental latex product, a dental non-latex product, or a dental cotton product.

The organosilane may be disposed on a dental product with or without the wetting agent. In some embodiments, the dental product is a saliva ejector, which typically is a narrow tube that dentists and other oral health care professionals use to suction saliva, blood and debris from the mouth during a dental visit. A saliva ejector may sit in the mouth during a dental procedure, such as filling teeth, or the dentist may insert it into the mouth at intervals during the procedure.

In some embodiments, the dental product is an articulating paper, which typically is a paper treated with a dye or wax that marks the points of contact made by the teeth when a patient bites or grinds on it. In some embodiments, the dental product is a dental dam, which can be latex or non-latex barrier used in dental procedures to isolate the tooth.

The antimicrobial substrate can be used in a variety of commercial and institutional applications including healthcare and non-healthcare settings. These include contract (professional) cleaning and/or disinfecting services, office facilities, corporations, banks, stock exchange, food and beverage industries, farms (e.g., chicken farms that may have Avian Flu present), hotel/restaurant/entertainment facilities, healthcare facilities (e.g., hospitals, urgent care facilities, clinics, nursing homes, hospices, medical/dental offices, laboratories), educational facilities, recreational facilities (e.g., arenas, coliseums, resorts, halls, stadiums, cruise lines, arcades, convention centers, museums, theatres, clubs, family entertainment complexes (indoor and/or outdoor), marinas, parks), food service facilities, governmental facilities, and public transportation areas (e.g., airports, airlines, cabs, buses, trains, subways, boats, ports, and their associated properties), ambulances, and/or cars. The antimicrobial substrate can be used for infection prevention and control of the spread of microorganisms wherever people congregate.

In various embodiments, an antimicrobial face mask is provided. The face mask can be disposable and/or for single use. Face masks are used as barriers between the wearer and the environment. Through their filtration efficiency, face masks can remove or trap particulates (organic, inorganic, or microbiological) from the incoming or outgoing breath. Face masks are generally not antimicrobially active even though they are commonly used in a healthcare setting as a method of minimizing pathogen transmission risk. However, the present face masks have antimicrobial activity, that is a mask capable of killing microorganisms that come into contact with it. In various embodiments, the killing of microorganisms occurs on contact, in seconds (e.g., 10, 20, or 30 seconds) or within minutes. This activity extends to antimicrobial kill of organisms like bacteria, fungi, and viruses, e.g., HIV (human immunodeficiency virus), hepatitis B virus (HBV), influenza A virus, the rhinovirus, hepatitis C virus (HCV), tuberculosis, Avian influenza, SARS, Staphylococcus aureus including methicillin resistant Staphylococcus aureus, (MRSA), Streptococcus species, vancomycin-resistant enterococci (VRE), Escherichia coli, Klebsiella pneumoniae, Pseudomonas aeruginosa, Fusarium species, Salmonela species, Shigella species, Yersinia species, Bacillus species, Norwalk virus, Campylobacteria, Clostridium botulinum, C. perfringes, Listeria monocytogenes, Penicillium, Aspergillus, and other pathogens.

In various embodiments, the microbes that the substrate inhibits growth of or kills the organisms in Table A.

TABLE A

Fungi
Aspergillus niger
Aspergillus fumigatus
Aspergillus versicolor
Aspergillus flavus
Aspergillus terreus
Penicillium chrysogenum
Penicillium albicans
Penicillium citrinum
Penicillium elegans
Penicillium funiculosum
Penicillium humicola
Penicillium notatum
Penicillium variabile
Mucor sp.
Tricophyton mentagrophytes
Tricophyton interdigitalie
Trichoderma flavus
Chaetomium globusum
Rhizopus nigricans
Cladosporium herbarum
Aureobasidium pullulans
Fusarium nigrum
Fusarium solani
Gliocladium roseum
Oosopa lactis
Stachybotrys chartarum
Algae
Oscillatoria borneti LB143
Anabaena cylindrica B-1446-1C
Selenastrum gracile B-325
Pleurococcus sp. LB11
Schenedesmus quadricauda
Gonium sp. LB 9c
Volvox sp. LB 9
Chlorella vulgarus
Yeast
Saccharomyces cerevisiae
Candida albicans
1. Clinical isolates
Stapylococcus epidermidis1
Enterobacter agglomerans1
Acinetobacter calcoaceticus1
Stapylococcus aureus(pigmented)1
Stapylococcus aureus (non-pigmented)1
Klebsiella pneumoniae ATCC 4352
Pseudomonas aeruginosa
Pseudomonas aeruginosa PRD-10
Strepticoccus faecalis
Pseudomonas aeruginosa1
Escherichia coli ATCC 23266
Escherichia coli1
Proteus mirabilis
Citrobacter diversus1
Salmonella typhosa
Proteus mirabilis1
Salmonella choleraesuis
Corynebacterium bovis
Mycobacterium smegmatis
Mycobacterium tuberculosis
Bruncella cania
Brucella abortus
Brucella suis
Streptococcus mutans
Bacillus subtilis
Bacillus cereus
Clostridium perfringens
Haemopilus influenzae
Haemophilus suis
Lactobacillus casei
Leuconostoc lactis
Listeria monocytogenes
Propionbacterium acnes
Proteus vulgaris
Pseudomonas cepacia
Pseudomonas filluorescens
Xanthomonas campestris
Virus
Influenza A
Avian Flu
HIV B
SARS

In various embodiments, the face mask comprises one or more layers individually or combined made of medical grade tissue, spun bound polypropylene, cellulose material, meltblown polypropylene, spun bound high density polyethylene, and/or low density polyethylene.

In various embodiments, the antimicrobial agent may be disposed on the outermost layer of the face mask. In various embodiments, the antimicrobial agent may also be advantageously located on a tissue, which is added to the face mask as an additional layer.

In various embodiments, one or more layers of the mask may be impervious or substantially impervious to liquid (e.g., spun bound polypropylene, and/or meltblown polypropylene layer(s)), which may cause an antimicrobial liquid to bead on one or more surfaces or layers of the mask. In such case, in this embodiment, the antimicrobial will not adhere to or be dispersed on the surface of the mask. There will be areas of the mask that will not be able to kill the microbe as little or no antimicrobial will be present. In various embodiments, depending on the surface, a wetting agent is used to modify the characteristics of the contact between the liquid antimicrobial and the surface to promote more rapid wetting of the substrate surface and more adherence of the antimicrobial to the surface of the substrate. In various embodiments, when the wetting agent is added, the antimicrobial has improved killing (e.g., 99.9% or 100% of microorganisms that it comes into contact with).

In various embodiments, for liquid impervious or non-porous substrates or substantially liquid impervious or non-porous substrates, the surface of the substrate is treated to improve the surface tension of the substrate to make it more receptive to the antimicrobial and allow it to adhere more to the substrate. By “porous” it is meant that one or more surfaces of the substrate are permeable to liquid or moisture. By “non-porous” it is meant that one or more surfaces of the substrate are impermeable to liquid or moisture.

Corona treatment of the substrate is one method to improve adhesion of the antimicrobial to the substrate. In general, corona treatment involves exposing the substrate to an electrical discharge, or “corona”, where oxygen molecules within the discharge area break into their atomic form and are free to bond to the ends of the molecules in the substrate being treated, resulting in a chemically activated surface that allows the antimicrobial to adhere more to the substrate. Other technologies used for surface treatment include in-line atmospheric (air) plasma, flame plasma, and chemical plasma systems.

In general, atmospheric plasma is very similar to corona, yet there are a few differences between them. Both types of treatment use one or more high voltage electrodes, which positively charge the surrounding blown air ion particles. However, in atmospheric plasma treatments, the rate at which oxygen molecules bond to a material's molecular ends occurs many more times. From this increase of oxygen, a higher ion bombardment occurs. This results in stronger material bonding traits and increased reception for coatings. Atmospheric plasma treatments also eliminates a possibility of treatment on a material's non-treated side; also known as backside treatment.

In general, flame plasma treatment can also be used to surface treat the substrate. Flame plasma treatment generates more heat than other treating processes, but materials treated through this method tend to have a longer shelf-life. Plasma treatment is different than air plasma treatment because flame plasma occurs when flammable gas and surrounding air are combusted together into an intense blue flame. The surface of the substrate is polarized from the flame plasma affecting the distribution of the surface's electrons in an oxidation form, which allows better bonding to the substrate.

Chemical plasma treatment can also be used to surface treat the substrate. Chemical plasma treatment is based on the combination of air plasma and flame plasma. Much like air plasma, chemical plasma fields are generated from electrically charged air. But, instead of air, chemical plasma relies on a mixture of other gases depositing various chemical groups onto the treated surface.

In various embodiments, one or more layers of a substrate (e.g., liner) can be surface treated by corona treatment, in-line atmospheric (air) plasma, flame plasma, and/or chemical plasma systems to make the antimicrobial more easily adhere to the substrate. For example, when a combination of HDPE and LDPE are used, one or both surfaces of the one or more layers of the substrate can be corona surface treated to improve wetting of the surface with the antimicrobial and adherence of the antimicrobial to the substrate. In various embodiments, from 1.25% to about 2.5% w/w or w/v of organosilane in water comprises an effective antimicrobial amount to apply or impregnate the substrate without the wetting agent.

In various embodiments, the one or more layers of a substrate may comprise a blend of HLDPE and the substrate is corona treated at a level of about 20 to about 100 dyne to allow the organosilane to adhere to the substrate. In various embodiments, about 1.25% to about 2.5% w/w or w/v of organosilane in water is applied to or impregnate on one or more surfaces of the substrate. After the organosilane is applied, one or more surfaces of the substrate are dried.

In various embodiments, the antimicrobial substrate is effective for killing at least 90%, in various embodiments, 99.9% of microorganisms that it comes into contact with. In various embodiments, 100% of the microorganisms are killed.

In various embodiments, the compositions are prepared by mixing the reagents (e.g., organosilane, stabilizer, water, and/or wetting agent) in any order. Typically, the compositions are generally prepared by first obtaining several mixing vats in which intermediate compositions are prepared that, when further reacted, form the antimicrobial composition. The mixing vats should be dry and free of water and solvents. Typically, the organosilane is made first, then diluted with a suitable solvent (e.g., water), then optionally, a wetting agent is added.

In various embodiments, stock solution of 500 mL of 5% w/v of 3-(trihydroxysilyl)propyldimethyloctadecyl ammonium chloride, 0.8% 3-chloropropyltrimethoxysilane, and 1.9% pentaerythritol (Goldshield™ 5 NBST 05) diluted in 500 mL of water is made and to this 1 L solution is added 100 mL of a nonionic wetting agent comprising ethoxylated alcohol <5% and glycol ethers, category N230 at 0.1%. The final solution is applied to the substrate. In various embodiments, there is no need to pre-treat the substrate. In other embodiments, the substrate can be pre-treated.

Once the antimicrobial composition is prepared, the substrate can be coated, sprayed, dipped, printed, and/or impregnated, with the composition.

In various embodiments, online spraying of the substrate is employed, which allows continuous and batch processes of substrates to be made of different colors, shapes and designs.

In various embodiments, the organosilane is applied to the substrate using an online spray system, which comprises a sprayer and a detection system that detects the distribution of the organosilane on the surface of the substrate. The spray system insures that the proper spray angle, plume geometry, spray pressure, and/or spray pattern is applied to the substrate and that organosilane contacts the edges of the substrate. Clogs in the sprayer may also be detected. One example of a detection method includes a laser scanner that scans the substrate to detect voids or spaces where there is no organosilane applied. The system may also include CCD cameras and/computers used to assist in visually detecting the spraying process.

In various embodiments, after contacting the substrate, the antimicrobial composition is dried. In various embodiments, drying can be accomplished by forced air at approximately 30 to 165° C. to remove surface residual solvent and debris or with heat lamps. In various embodiments, the drying can occur in free air at room temperature.

In various embodiments, after the antimicrobial is applied to the substrate, the substrate is dried to insure proper distribution of the antimicrobial on the surface of the substrate. This, among other things, prevents the antimicrobial from migrating off the substrate and is particularly important when dealing with water-impervious substrates, such as for example, face masks. In various embodiment, the substrate can be dried at 30 to 200° C. using forced air, heat lamps, and/or rollers leading into a hot box, so that the substrate is completely dry and the antimicrobial will not migrate on the substrate. Thus, there will not be spots or patches where no antimicrobial is present—which will undesirably allow microbes to grow.

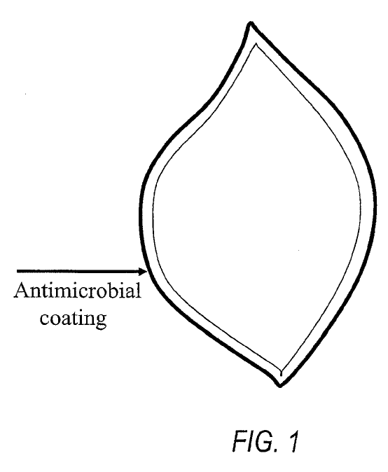

FIG. 1 illustrates a typical substrate on which the organosilane is applied. In this illustration a face mask for a medical, dental, surgical use or non-healthcare use is shown. An antimicrobial amount of the organosilane is applied to the outer surface or layer of the mask that faces the outside environment away from the user.

In various embodiments, the substrate is treated with the antimicrobial composition by, for example, coating, spraying, dipping, impregnating the composition into or on the substrate. The treatment can be done, for example, by passing continuous or batch rolls of the substrate so that the antimicrobial composition contacts the substrate. This can be accomplished at room temperature or using heat or pressure rollers.

In various embodiments, the substrate can be submerged in the treatment solution and rotated through, causing said solution to contact the substrate. Thereafter, the treated substrate containing the measured volume of treatment solution may be wound onto rolls and/or converted into the desired product. For the purposes of this specification, the term “conversion” means the process(es) of modifying the physical characteristics of the treated substrate by such conventional methods as embossing, laminating, slitting, and cutting so that the treated matrix is rendered into a form that is saleable as a manufactured product and is ready for distribution. Other methods of impregnating the matrix with measured amounts of treatment solution, such as by spraying, dipping, extrusion or by reverse roll, may also be used. The coating/spraying, submerging, and /or impregnation methods enable a uniform and accurate application of all active ingredients to the woven or nonwoven substrate of natural and/or synthetic fibers.

In various embodiments, when the composition comprises an organosilane (e.g., 3-(trihydroxysilyl)propyl-dimethyloctadecyl ammonium chloride), it binds to the surface either through (i) ionic bonds between O on a negatively charged surface possessing acidic hydroxyl groups and the positively charged ammonium ion, through (ii) covalent bonds between OH on a surface possessing non-acidic hydroxyl groups and the —Si—OH group, or through (iii) electrostatic attraction between the negative charge that exist on most non-hydroxylated surfaces and the positively charged ammonium ion. It is also believed that intermolecular siloxane polymerization (—Si—O—Si— bonds) occurs on the surface between the surface-associated molecules.

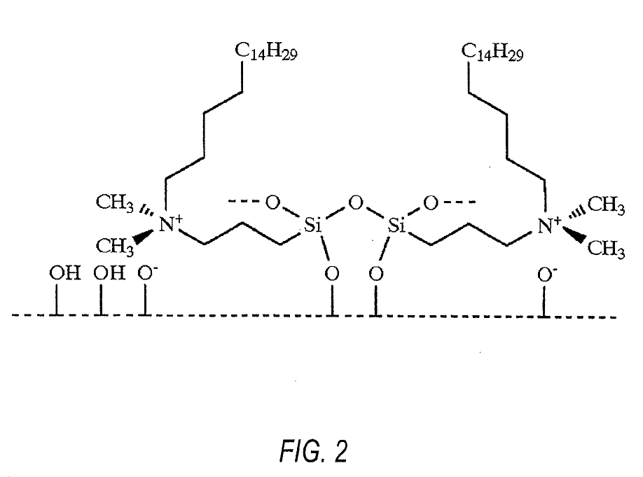

FIG. 2 illustrates a view of a substrate, in this case, non-woven polymer strands having the antimicrobial composition coated on the surface of the polymer. In this illustrated embodiment, some of the polymer surface has an antimicrobial amount of the organosilane applied to it. It will be understood that some, all, or substantially all of the polymer surface will have the antimicrobial applied to it.

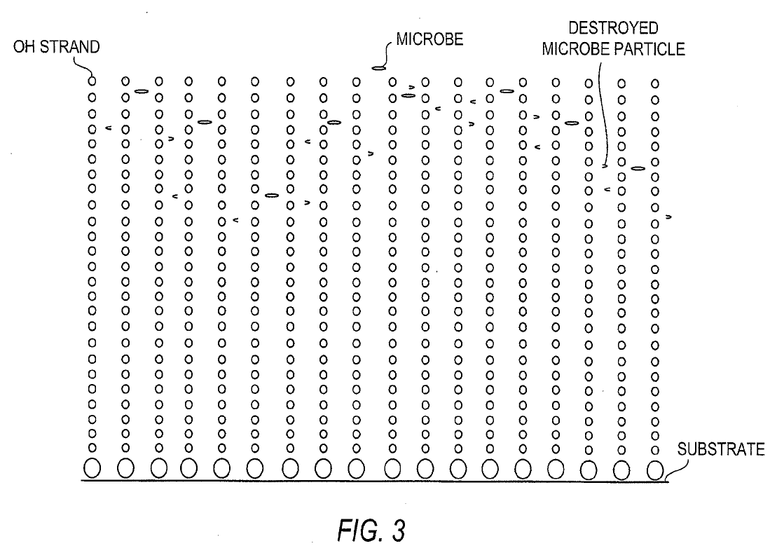

FIG. 3 illustrates a polymer surface having the organosilane applied to the surface, where the organosilane associates with the surface of the substrate through, for example, ionic bonding, covalent bonding, hydrogen bonding, electrostatic interaction, hydrophobic, hydrophilic or other interaction with the surface of the substrate. The antimicrobial activity in this illustration is exerted when the microbe usually having a negative charge is attracted to the positively charged nitrogen, when the microbe comes into contact with the carbon chain, the microbe is killed or the growth of the microbe is inhibited.

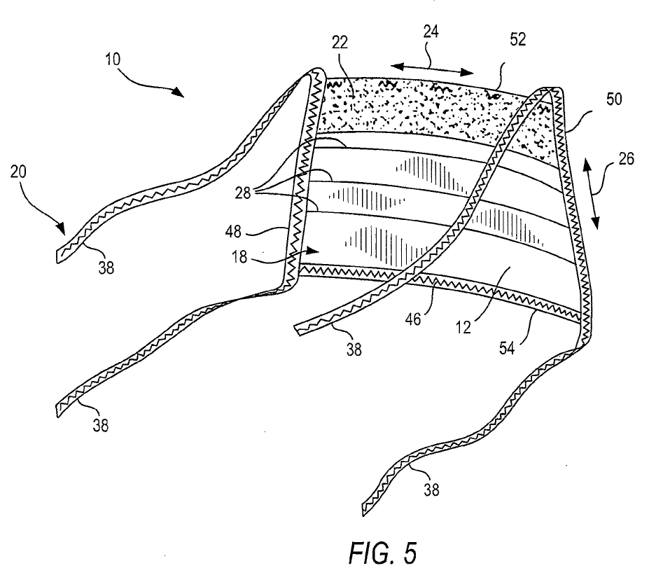

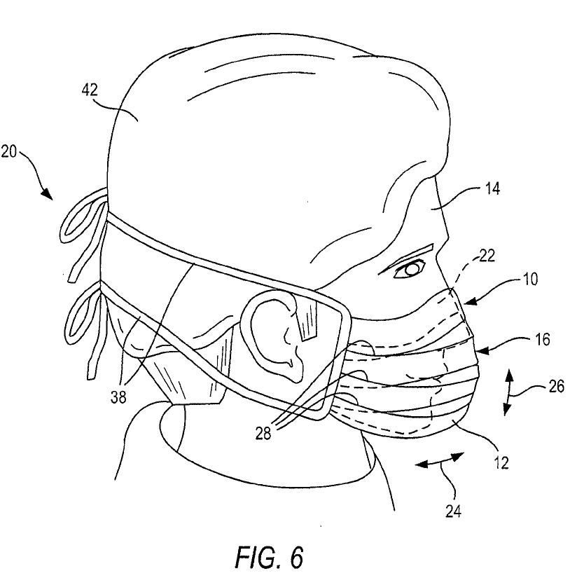

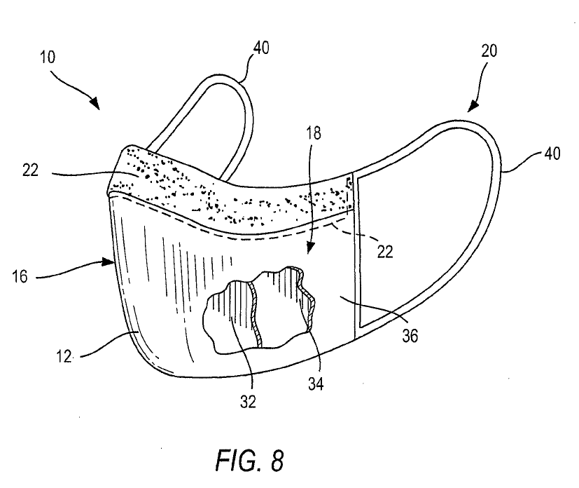

One exemplary embodiment, shown in FIG. 8, provides a face mask 10 that has a body portion 12 with an outer surface 16 and an oppositely disposed inner surface 18. FIG. 5 illustrates a perspective view of the face mask 10 in accordance with one exemplary embodiment. The face mask 10 includes a body portion 12 that has an inner surface 18 that is configured for facing and contacting the face of a user 14 (FIG. 6) when the face mask 10 is worn. The mask may include a plurality of folds 28 and may also include a malleable member 22. The malleable member 22 is provided so that the top binding 52 of the body 12 can be configured to closely fit the contours of the nose and upper cheeks of the wearer. The malleable member 22 is preferably constructed from a metal strip with a rectangular cross-section, but may be a moldable or a malleable metal or alloy, plastic, or any combination thereof.

FIG. 6 illustrates a perspective view of the face mask 10 of FIG. 5 shown attached to the face of the user 14. The body portion 12 can be of a variety of styles and geometries, such as, but not limited to, flat half masks, pleated face masks, cone masks, duckbill style masks, trapezoidally shaped masks, etc. The styles shown in the Figures are for illustrative purposes only. As shown in FIG. 6, the face mask 10 may isolate the mouth and the nose of the user 14 from the environment. The face mask 10 may be attached to the user 14 by a fastening member 20 that may be a pair of manual tie straps 38 that are wrapped around the head of the user 14 (and a hair cap 42 if worn by the user 14) and are connected to one another. It is to be understood, however, that other types of fastening members 20 may be employed in accordance with various exemplary embodiments. Instead of the manual tie straps 38, for example, the face mask 10 may be attached to the user 14 by a fastening member 20 that may be ear loops 40 (FIG. 8), elastic bands wrapped around the head of the user 14, a hook and loop type fastener arrangement (e.g. VELCRO® fasteners), or a connection directly attaching the face mask 10 to the hair cap 42.

Additionally, the configuration of the face mask 10 is different in accordance with various exemplary embodiments. In this regard, the face mask 10 can be made in order to cover both the eyes, hair, nose, throat, and mouth of the user 14. As such, the present application includes face masks 10 that cover areas above and beyond simply the nose and mouth of the user 14. The face mask 10 may also incorporate any combination of known face mask 10 features, such as visors or shields, sealing films, beard covers, etc.

The body portion 12 of the face mask 10 may be made of inelastic materials. Alternatively, the material used to construct the body portion 12 may be comprised of elastic materials, allowing for the body portion 12 to be stretched over the nose, mouth, and/or face of the user 14. (FIG. 6) The face mask 10 of the present application may be made of an elastic material that allows the face mask 10 to stretch in one or more directions. The use of an elastic material incorporated into the body portion 12 may allow for fuller coverage of the user's 14 face and provide for more flexibility in accommodating variously sized faces of the users 14. Alternatively, the body portion 12 may be made of an inelastic material. As such, the material that makes up the face mask 10 may exhibit elastic or inelastic characteristics depending upon the user's 14 needs.

The body portion 12 of the face mask 10 may be configured so that it is capable of stretching across the face of the user 14 (FIG. 6) from ear to ear and/or nose to chin. The ability of the body portion 12 to stretch and recover may provide the face mask 10 with better sealing capabilities and a more comfortable fit than face masks 10 that have an inelastic body portion 12. In order for the body portion 12 to stretch and recover, the body portion 12 may have at least one layer or a material that has stretch and recovery properties. Additionally, the entire face mask 10 may be composed of a material that has stretch and recovery properties in other exemplary embodiments. In certain exemplary embodiments, the percent recovery is about 15% and the percent stretch is between about 15-65%, in other embodiments the percent recovery is between about 20-40% stretch, and in still other embodiments the percent recovery is between about 25-30% stretch.

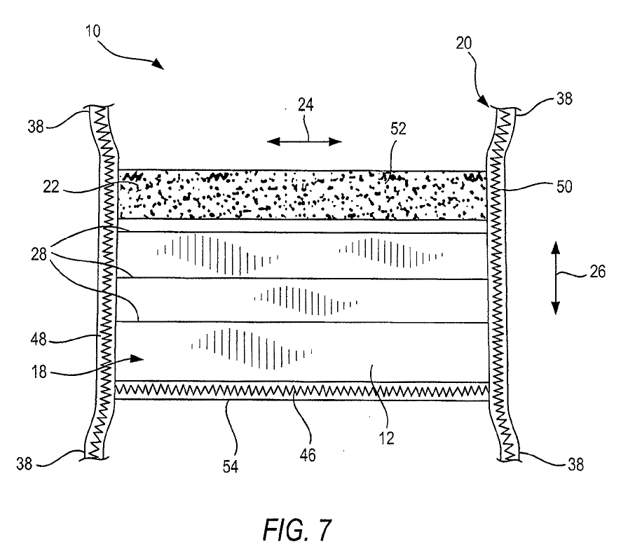

FIG. 7 shows the inner surface 18 of the body portion 12 of an exemplary embodiment of the face mask 10. The body portion 12 is typically provided with a plurality of folds 28 that extend across the horizontal length 24. The folds 28 may be opened by the user 14 so as to adjust the size of the body portion 12 in the vertical length 26. The folds 28 allow for adjustment of the body portion 12 so as to allow for a better fit on the face of the user 14 and formation of a breathing chamber. The body portion 12 may form a breathing chamber with the perimeter of the chamber sealing to the face of the user 14 in certain exemplary embodiment. Although shown as extending across the entire horizontal length 24, the folds 28 may extend only part way across the body portion 12. Additionally, the folds 28 may be provided in any number and may be oriented at any angle on the body portion 12. For instance, the folds 28 may run at a 45 degree angle to the horizontal length 24. Alternatively or additionally, the folds 28 may run along the vertical length 26 of the body portion 12. It is to be understood, however, that folds 28 are not required to be present in the practice of the invention and are merely shown for illustrative purposes.

Bindings (FIGS. 5 and 7) 48 and 50 may act to limit the vertical extension of the edges of the body portion 12 when the folds 28 are unfolded. As such, bindings 48 and 50 may be present in order to help provide for a desired shape of the body portion 12. Additionally, bindings 52 and 54 may also act to limit extension of the edges of the body portion 12 when folds 28 are unfolded. This may also be the case if folds 28 are provided in orientations along both the horizontal and vertical lengths 24 and 26 of the body portion 12. As such, bindings 52 and 54 may also be employed in order to achieve a desired shape of the body portion 12.

The folds 28 in the body portion 12 may be of any type commonly known to those having ordinary skill in the art. FIG. 7 shows bindings 48 and 50 on either side of the body portion 12 used in order to constrain unfolding of the folds 28. Additionally, binding 52 may be located on the top edge of the body portion 12 and binding 54 may be located on the bottom edge of the body portion 12. The bindings 48, 50, 52 and 54 may be of any type commonly known to one having ordinary skill in the art.

In FIG. 8, the body portion 12 is shown as being made of a plurality of layers 32, 34 and 36. Layer 32 may be an inner layer of the body portion 12 and have the inner surface 18 defined thereon. Layer 36 may be a protective, on-filtration outer layer of the body portion 12 and have the outer surface 16 defined thereon. Further, layer 34 may be an intermediate layer located between the layers 32 and 36. It is to be understood, however, that the body portion 12 may be made of any number of layers in accordance with the teachings herein. For instance, the body portion 12 can be made of a single layer in accordance with one exemplary embodiment. Alternatively, the body portion 12 can be made of three layers, five layers, seven layers, ten or fifteen layers in accordance with various embodiments. It will be understood that one or more layers may be coated, sprayed, dipped, printed, and/or impregnated, with the antimicrobial composition.

The intermediate layer 34, as shown in FIG. 8, may be a filtration media configured to prevent the passage of pathogens through the body portion 12 while still allowing for the passage of air in order to permit the user 14 (FIG. 6) to breath.

The layers 32, 34 and 36 may be constructed from various materials known to those skilled in the art. Layer 36 of the body portion 12 protects the inner filtration medium from physical damage and may be any nonwoven web, such as a spunbonded, meltblown, or coform nonwoven web, a bonded carded web, or a wet laid polyester web or wet laid composite. The layer 36 of the body portion 12 and layer 32 may be a necked nonwoven web or a reversibly necked nonwoven web. The layers 32, 34 and 36 may be made of the same material or of different materials. A tissue layer (not shown) may be located subjacent the outer most layer of the face mask.

Many polyolefins are available for nonwoven web production, for example polyethylenes such as Dow Chemical's ASPUN® 6811A linear polyethylene, 2553 LLDPE and 25355, and 12350 polyethylene are such suitable polymers. Fiber forming polypropylenes include, for example, Exxon Chemical Company's Escorene® PD 3445 polypropylene and Basell's PF-304. Many other suitable polyolefins are commercially available as are known to those having ordinary skill in the art. Other thermoplastic resins can also be used and include polyester, nylon, polylactic acid, polyglycolic acid and copolymers thereof, fluorinated thermoplastic resins such as inherently fluorinated polyethylene-co-polypropylene (FEP), polyvinylidene fluoride (PVDF) and the like.

The various materials used in construction of the face mask 10 may include a necked nonwoven web, a reversibly necked nonwoven material, a neck bonded laminate, and elastic materials such as an elastic coform material, an elastic meltblown nonwoven web, a plurality of elastic filaments, an elastic film, or a combination thereof. In an exemplary embodiment where an elastic film is used on or in the body portion 12, the film may be sufficiently perforated to ensure that the user 14 (FIG. 6) can breathe through the body portion 12 if the face mask 10 is desired to be breathable in this location.

The intermediate layer 34 is configured as a filtration layer and may be a meltblown nonwoven web and, in some embodiments, is electret treated. Electret treatment results in a charge being applied to the intermediate layer 34 that further increases filtration efficiency by drawing particles to be filtered toward the intermediate layer 34 by virtue of their electrical charge.

The intermediate layer 34 may be made of an expanded polytetrafluoroethylene (PTFE) membrane, such as those manufactured by W. L. Gore & Associates. A more complete description of the construction and operation of such materials can be found in U.S. Pat. Nos. 3,953,566 and 4,187,390 to Gore, the entire contents of these patents are incorporated herein by reference in their entirety for all purposes. The expanded polytetrafluoroethylene membrane may be incorporated into a multi-layer composite, including, but not limited to, an outer nonwoven web layer 36, an extensible and retractable layer, and an inner layer 32 comprising a nonwoven web.

SMS may be used to comprise the layers 32, 34 and 36. SMS is a material that is made of meltblown fibers between two spunbond layers made of spunbonded fibers. Spunbonded fibers are small diameter fibers, which are formed by extruding molten thermoplastic material as filaments from a plurality of fine, usually circular capillaries of a spinneret with the diameter of the extruded filaments then being rapidly reduced to fibers by methods known in the art. Spunbond fibers are generally continuous and have diameters generally greater than about 7 microns, more particularly, between about 10 and about 40 microns. Meltblown fibers are fibers formed by extruding a molten thermoplastic material through a plurality of fine, usually circular, die capillaries as molten threads or filaments into converging high velocity, usually hot, gas (e.g. air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly disbursed meltblown fibers. In various embodiments, meltblown fibers are microfibers, which may be continuous or discontinuous with diameters generally less than 10 microns.

Multiple layers of the face mask 10 may be joined by various methods, including adhesive bonding, thermal point bonding, or ultrasonic bonding. Although shown as having three layers 32, 34 and 36, it is to be understood that in other exemplary embodiments of the present invention, that the body portion 12 and/or the entire face mask 10 may be made of any number of layers.

While antimicrobial treatment may be applied to all types of face masks, surgical and infection preventions and control face masks are particularly useful. Surgical and infection preventions and control face masks may have a bacterial filtration efficiency (BFE) of greater than or equal to about 85-90% as measured according to ASTM F2101. More particularly, the mask exhibits a BFE of greater than or equal to about 95%. Still more particularly, the mask possesses a BFE of greater than or equal to about 99%. The face mask may exhibit a differential pressure less than or equal to 8 mm water/cm² as measured by ASTM F2101 to ensure the respiratory comfort of the product. Desirably, the differential pressure is less than or equal to 5 mm water/cm² and more desirably less than or equal to 2.5 mm water/cm². The face mask can have a particle filtration efficiency (PFE) of greater than or equal to about 85-90% as measured by Latex Particle Challenge testing (ASTM F2299). More particularly, the PFE is greater than or equal to 95%. Still more particularly, the PFE is greater than or equal to 99%. The face mask may be impervious to liquids and have a fluid penetration resistance of greater than or equal to about 80 mm Hg against synthetic blood as measured according to ASTM F1862. More particularly, the mask exhibits a fluid penetration resistance of greater than or equal to about 120 mm Hg. Still more particularly, the mask exhibits a fluid penetration resistance of greater than or equal to about 160 mm Hg.

The antimicrobial composition optionally may be present on the outermost layer of the face mask, not the filtration layer. Locating the antimicrobial composition on the outermost layer provides the additional benefit of reducing the contact transfer of microbes, in addition to reducing their passage through the mask. Furthermore, the location of the antimicrobial composition on the outer layer of the mask reduces the possibility that the antimicrobial composition will pass through the mask and be inhaled by a wearer. It will be understood that the antimicrobial can be disposed on one or more layers of the substrate and on one or both sides of the substrate on each layer.

Having now generally described the invention, the same may be more readily understood through the following reference to the following examples, which are provided by way of illustration and are not intended to limit the present invention unless specified.

### EXAMPLES

### Example 1

### Preparation of Water Stabilized Organosilane.

5% w/v (weight/volume) aqueous solution of 3-(trimethoxysilyl)propyldimethyloctadecyl ammonium chloride may be prepared using the procedure described in U.S. Pat. No. 6,221,994, col. 37-38 (however, no wetting agent is disclosed) as follows:

a) A 22 L reaction flask may be charged with 6250 g. (21.0 Mol.) of dimethyloctadecylamine, 5844 g. (29.4 Mol.) of 3-chloropropyltrimethoxysilane, and 76 g. (0.84 Mol.) of trioxane.

b) The mixture may be heated to 140° C. for 12 hours while stirring and is then may be cooled to 80° C.

c) 2 L of methanol may then be added and the mixture may be cooled to approximately 40° C.

d) This mixture may then be transferred to 171 L of water, into which 4000 g. of pentaerythritol may be previously dissolved. After thorough mixing the pH of the solution may be checked. If the pH is above 7.0 (basic) a small amount of HCl may be added until the pH is below 7.0.

e) The mixture may then be diluted to 209 L with additional water. The resulting solution will contain approximately 5% 3-(trihydroxysilyl)propyldimethyloctadecyl ammonium chloride, 0.8% 3-chloropropyltrimethoxysilane, and 1.9% pentaerythritol. The pentaerythritol stabilized 3-(trihydroxysilyl)propyldimethyloctadecyl ammonium chloride is soluble in water up to concentrations of approximately 15% w/v. This mixture may then be diluted for use to one part 5% active to any of the following to get desired efficacy: 4 parts water; 6 parts water; 7 parts water; 9 parts water.

f) In formulations where a wetting agent is used. To effectively and efficiently apply to inert materials such as polypropylene, prior to dilution one may add 10% nonionic wetting agent consisting primarily of: Ethoxylated alcohol <5% and glycol ethers, category N230 at 0.1%. The resulting formula diluted seven (7) parts water will be 1.0% active; 1.42% wetting agent; and balance water.

### Example 2

Comparison of Untreated Masks, 5% Water Stabilized Organosilane Treated Masks With or Without Wetting Agents

Preparation: Three masks tested and results are listed in Table 1. Dark blue indicates where the mask was treated with a solution of 5% (w/v) 3-(trihydroxysilyl)-propyldimethyloctadecyl ammonium chloride, 0.8% 3-chloropropyltrimethoxysilane, and 1.9% pentaerythritol in water referred to as 5% concentrated of the pentaerythritol stabilized 3-(trihydroxysilyl)propyldimethyloctadecyl ammonium chloride. This organosilane may be prepared using the procedure described in Example 1. Light blue indicates where the mask was treated with a solution of 5% 3-(trihydroxysilyl)propyldimethyloctadecyl ammonium chloride, 0.8% 3-chloropropyltrimethoxysilane, and 1.9% pentaerythritol referred to as 5% concentrated of the pentaerythritol stabilized 3-(trihydroxysilyl)propyldimethyloctadecyl ammonium chloride. The wetting agent was used at a concentration of 0.1% w/v of a 10% nonionic wetting agent containing primarily of ethoxylated alcohol <5% and remainder glycol ethers. Pink indicates that the mask was untreated.

Each mask was cut into four equal parts. The elastic bands on the dark and light blue masks were removed, though some stitching still remained. There was no band on the untreated mask. Two sections from each mask were identified as replicates A and two were identified as replicates B. Replicates A were placed into each of three sterile culture containers. B replicates were also placed into each of three sterile culture containers for a total of six separate cultures. Each culture container held two mask sections, one to be used for the 2 hour time point and one to be used for the 24 hour time point.

A methicillin resistant Staphylococcus aureus patient isolate was grown overnight in a muehler-hinton liquid broth culture. Titer was determined by optical density and liquid culture was diluted to a concentration of 1×10⁷ organisms per mL. Mask material in each culture container was inoculated with 5×10⁷ organisms. All liquid was absorbed by the material, though total absorption took longer for the dark blue and pink masks. Masks were incubated at 35° C. without CO₂. At 2 hours, one mask section from each of the six containers was selected and placed into sterile jars into which 100 mL of sterile saline was added. The jars were vigorously shaken for approximately 2 minutes. Aliquots of the saline solution were taken and dilutions were made for 10⁰, 10¹ and 10². 100 microL from each dilution was plated as a lawn on to TSA plates containing 5% sheep blood. Plates were incubated at 35° C. without CO₂ for 24 hours and colony-forming units were enumerated. Plates were placed back into incubation for a further 24 hours for a total of 48 hours and colony-forming units were enumerated again.

At 24 hours post inoculation, the remaining mask sections were removed from each of the six containers and sampled the results are below.

TABLE 1
Results

| | 2 h | | | Av- | 24 h | | | Av- |
|---|---|---|---|---|---|---|---|---|
| Mask | dilution | 24 h | 48 h | erage | dilution | 24 h | 48 h | erage |
| Untreated | 1 | TNC | TNC | | 1 | TNC | TNC | |
| rep A | 10 | TNC | TNC | | 10 | TNC | TNC | |
| | 100 | 600 | TNC | | 100 | 1320 | TNC | |
| | 1 | TNC | TNC | | 1 | TNC | TNC | |
| rep B | 10 | TNC | TNC | | 10 | TNC | TNC | |
| | 100 | 1500 | TNC | 1050 | 100 | 1836 | TNC | 1578 |
| 5% conc | | | | | | | | |
| | 1 | 0 | 0 | | 1 | 0 | 0 | |
| rep A | 10 | 0 | 0 | | 10 | 0 | 0 | |
| | 100 | 0 | 0 | | 100 | 0 | 0 | |
| | 1 | 1972 | TNC | | 1 | 12 | 12 | |
| rep B | 10 | 271 | TNC | | 10 | 4 | 4 | 2 |
| | 100 | 19 | 22 | 10 | 100 | 0 | 0 | |
| 5% + | | | | | | | | |
| wetting | | | | | | | | |
| | 1 | 0 | 0 | | 1 | 0 | 0 | |
| rep A | 10 | 0 | 0 | | 10 | 0 | 0 | |
| | 100 | 0 | 0 | | 100 | 0 | 0 | |
| | 1 | 0 | 0 | | 1 | 0 | 0 | |
| rep B | 10 | 0 | 0 | | 10 | 0 | 0 | |
| | 100 | 0 | 0 | 0 | 100 | 0 | 0 | 0 |

TNC = too numerous to count

At the 2 hour evaluation, the 5% concentrated product resulted in a reduction in bacterial load of 99.05%. The mask treated with the 5% solution plus the wetting agent resulted in a 100% reduction. The addition of the wetting agent made the compound more effective.

At the 24-hour evaluation, the 5% concentrated product reduced bacterial load by 99.99%. The mask treated with the 5% solution plus the wetting agent resulted in 100% reduction.

Reductions were calculated using the number of colonies obtained from blood plates after 24 hours of incubation.

### Example 3

Comparison of Untreated Masks, 1% Water Stabilized Organosilane Treated Masks With or Without Wetting Agents

Preparation: Three masks tested and results are listed in Table 1. Dark blue indicates where the mask was treated with a solution of 1% (w/v) 3-(trihydroxysilyl)-propyldimethyloctadecyl ammonium chloride, 0.8% 3-chloropropyltrimethoxysilane, and 1.9% pentaerythritol in water referred to as 1% concentrated of the pentaerythritol stabilized 3-(trihydroxysilyl)propyldimethyloctadecyl ammonium chloride. This organosilane may be prepared using the procedure described in Example 1. Light blue indicates where the mask was treated with a solution of 1% 3-(trihydroxysilyl)propyldimethyloctadecyl ammonium chloride, 0.8% 3-chloropropyltrimethoxysilane, and 1.9% pentaerythritol referred to as 1% concentrated of the pentaerythritol stabilized 3-(trihydroxysilyl)propyldimethyloctadecyl ammonium chloride. The wetting agent was used at a concentration of 0.1% w/v of a 10% nonionic wetting agent containing primarily of ethoxylated alcohol <5% and remainder glycol ethers. Yellow indicates that the mask was untreated.

Each mask was cut equally into four equal parts. The elastic bands from each mask were removed, though some stitching still remained. Two sections from each mask were identified as replicates A and two were identified as replicates B. Each section of the mask were placed into a separate sterile culture container (a total of 12 containers). A methicillin resistant Staphylococcus aureus isolate was grown in a muehler-hinton liquid broth culture. Titer was determined by optical density and liquid culture was diluted to a concentration of 5×10⁷ organisms per mL. Each section was inoculated with 5×10⁷ organisms per mL. All of the liquid was absorbed by the material.

Masks were incubated at 35° C. without CO₂. At 2 hours, one mask section from each of the different masks were placed into 100 mL of sterile saline. The containers were vigorously shaken for approximately 2 minutes. Aliquots of the saline solution were taken and dilutions were made for 10⁰, 10¹ and 10². 100 microL from each dilution was plated as a lawn on to Tryptic (Trypticase) Soy Agar (TSA) plates. Plates were incubated at 35° C. without CO₂ for 24 hours and colony-forming units were enumerated. Plates were placed back into incubation for a further 24 hours for a total of 48 hours and counting of colonies were performed again.

At 24 hours post inoculation, the remaining mask sections were removed from and sampled.

TABLE 2
Results

| | 2 h | | | Av- | 24 h | | | Av- |
|---|---|---|---|---|---|---|---|---|
| Mask | dilution | 24 h | 48 h | erage | dilution | 24 h | 48 h | erage |
| untreated | | | | | | | | |
| A | 1 | TNC | TNC | | 1 | TNC | TNC | |
| | 10 | 120 | TNC | | 10 | TNC | TNC | |
| | 100 | 75 | TNC | | 100 | TNC | TNC | |
| B | 1 | TNC | TNC | | 1 | TNC | TNC | |
| | 10 | 230 | TNC | | 10 | TNC | TNC | |
| | 100 | 105 | TNC | 132.5 | 100 | TNC | TNC | TNC |
| 1% conc | | | | | | | | |
| A | 1 | 1 | 1 | | 1 | TNC | TNC | |
| | 10 | 0 | 0 | | 10 | TNC | TNC | |
| | 100 | 0 | 0 | | 100 | TNC | TNC | |
| B | 1 | 6 | 10 | | 1 | TNC | TNC | |
| | 10 | 0 | 0 | | 10 | TNC | TNC | |
| | 100 | 0 | 0 | 4.5 | 100 | TNC | TNC | TNC |
| 1% + | | | | | | | | |
| wetting | | | | | | | | |
| A | 1 | 1 | 2 | | 1 | TNC | TNC | |
| | 10 | 0 | 0 | | 10 | TNC | TNC | |
| | 100 | 0 | 0 | | 100 | TNC | TNC | |
| B | 1 | 0 | 0 | | 1 | TNC | TNC | |
| | 10 | 0 | 0 | | 10 | TNC | TNC | |
| | 100 | 0 | 0 | 1.5 | 100 | TNC | TNC | 0 |

TNC = too numerous to count

At the 2 hour evaluation, the 1% concentrated product of the pentaerythritol stabilized 3-(trihydroxysilyl)propyldimethyloctadecyl ammonium chloride resulted in a reduction in bacterial load of 96.6%. The mask treated with the 1% solution plus the wetting agent resulted in a 98.87% reduction. The addition of the wetting agent made the compound more effective.

At the 24-hour evaluation, there was a 0% reduction in bacterial growth with the 1% concentrated product and 1% concentrated product.

Reductions were calculated using the number of colonies obtained from the TSA plates after 24 hours of incubation.

### Example 4

Efficacy Tests on 2.5% Organosilane Plus 5% Wetting Agent Applied to Dental Mask:

The dental mask was treated with a solution of 2.5% 3-(trihydroxysilyl)propyldimethyloctadecyl ammonium chloride, 0.8% 3-chloropropyltrimethoxysilane, and 1.9% pentaerythritol referred to as 1% concentrated of the pentaerythritol stabilized 3-(trihydroxysilyl)propyldimethyloctadecyl ammonium chloride (Goldshield™ 5 NBST 05). The wetting agent was used at a concentration of 0.1% w/v of a 5% nonionic wetting agent containing primarily of ethoxylated alcohol <5% and remainder glycol ethers.

Mask Test:

Preparation:

Two masks tested.

Control: containing No Goldshield™ product.

Mask A: Containing 2.5% Goldshield™ (3-(trihydroxysilyl)propyldimethyloctadecyl ammonium chloride) antimicrobial solution plus wetting agent.

The test was performed to determine in this embodiment the following:

Reduction of bacterial growth (MRSA), time it takes to render the bacteria ineffective, and proper distribution of the antimicrobial over the mask.

Procedure:

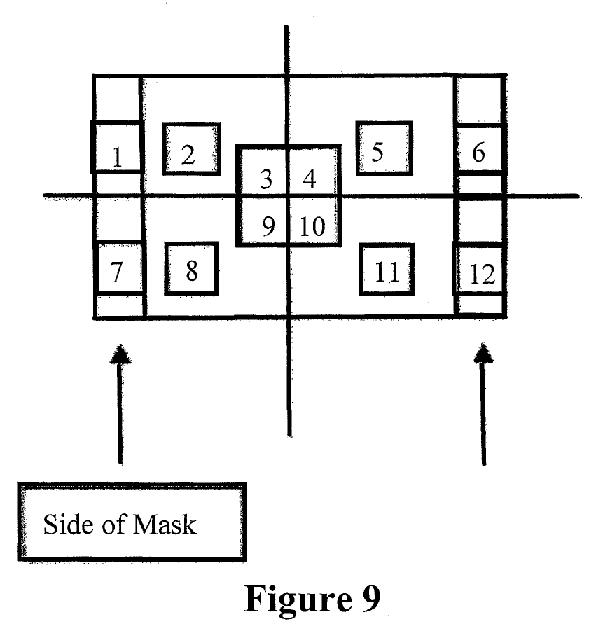

A total of 12 specific sections of the mask were cut into 0.5 square inches. 100 ul of 10⁷ cells was applied to each of the cut sections. This is an extremely high amount of bacteria (10⁷=10 million bacterial colonies) applied to the mask and will be several thousand to one million times more than will be encountered in healthcare and non-healthcare environments. The section of the mask was then placed on the surface of the TSA agar plate for the specific time point (1 min, 30 min, 1 hr., 2 hr., 4 hr., 24 hr.). After the time point, the mask section was lifted off the agar and the agar plate was placed in a 37° C. CO₂ incubator for 24 hr. FIG. 9 shows the mask sections tested.

Results:

Efficacy tests on 2.5% Goldshield™ Antimicrobial Solution+Wetting Agent Applied to Dental Masks

| | Colonies | | Colonies | | Colonies | | Colonies | | Colonies | |
|---|---|---|---|---|---|---|---|---|---|---|
| Mask | 1 minute | | 30 mins. | | 1 hour | | 4 hours | | 24 hours | |
| Section | Treated | Control | Treated | Control | Treated | Control | Treated | Control | Treated | Control |
| 1 | 8 | TMC | 0 | TMC | 0 | TMC | 0 | TMC | 0 | TMC |
| 2 | 44 | TMC | 0 | TMC | 0 | TMC | 1 | TMC | 0 | TMC |
| 3 | 27 | TMC | 0 | TMC | 0 | TMC | 0 | TMC | 0 | TMC |
| 4 | 21 | TMC | 0 | TMC | 0 | TMC | 0 | TMC | 0 | TMC |
| 5 | TMC | TMC | 1 | TMC | 0 | TMC | 0 | TMC | 0 | TMC |
| 6 | 128 | TMC | 0 | TMC | 0 | TMC | 0 | TMC | 0 | TMC |
| 7 | 3 | TMC | 0 | TMC | 0 | TMC | 0 | TMC | 3 | TMC |
| 8 | 44 | TMC | 0 | TMC | TMC | TMC | 0 | TMC | 0 | TMC |
| 9 | 60 | TMC | 0 | TMC | 0 | TMC | 0 | TMC | 0 | TMC |
| 10 | 79 | TMC | 0 | TMC | 0 | TMC | 0 | TMC | 0 | TMC |
| 11 | TMC | TMC | 0 | TMC | 0 | TMC | 0 | TMC | 0 | TMC |
| 12 | 10 | TMC | 13 | TMC | 0 | TMC | 0 | TMC | 0 | TMC |

TMC: Too many to count

Conclusion:

At one minute adherence with the mask sections, there is bacterial growth of MRSA. As time of mask adherence increased there is less of an amount or no bacterial growth on the agar plates.

Proper Distribution of the Antimicrobial Mask:

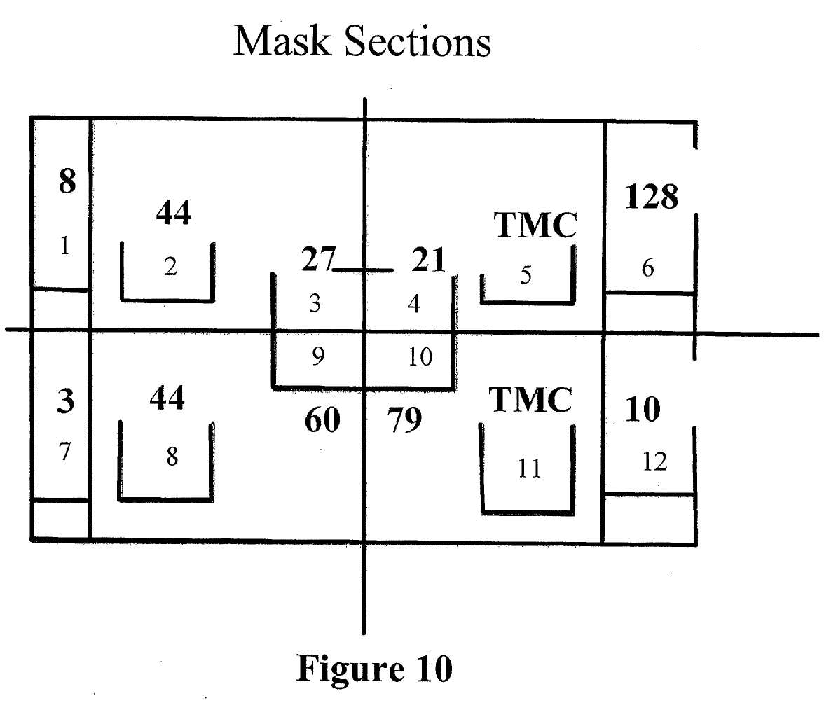

The boxes in FIG. 10 with the numbers inside indicate the mask section number and the numbers above indicate the amount of colonies at the one minute point. An extremely high amount of bacteria (10⁷=10 million bacterial colonies) were applied to the mask and will be several thousand to one million times more than will be encountered in healthcare and non-healthcare environments. As you can see, as you move from (top section) left to right of the mask, there is an increase in colonies at section 2, then a decrease at 3 and 4, and finally an increase at sections 5 and 6. On the bottom section, you see a similar pattern occurring, but at least the last mask section (12) there is dramatic decrease in colonies. Again out of the 10 million bacterial colonies that were implanted on the mask only very few bacterial colonies were left (a 99.999% kill) and the bacterial kill by the 2.5% Goldshield™ antimicrobial solution was surprising. In general, this example shows that the organosilane adheres to the mask and decreased or eliminated MRSA bacteria. It also shows, in various embodiments, that the proper distribution of the antimicrobial on the mask is important. Sections of the mask that have no antimicrobial applied to it (blank spots) due to antimicrobial migration should be avoided.

One way to insure proper distribution of antimicrobial on the mask and avoid migration of the antimicrobial (avoid blank spots) is to use a wetting agent and proper drying techniques. One can double check the batch by sampling the mask and applying a pH indicator such as bromophenol, which will turn blue (indicating a pH change) when the antimicrobial is applied to the mask.

### Example 5

Skin Irritation Study: 2.5% Goldshield™ Antimicrobial Solution Treated Face Mask

The test article, 2.5% Goldshield treated face mask, Batch: Test II Sep. 21, 2007, was evaluated for primary skin irritation in accordance with the guidelines of the International Organization for Standardization 10993. Biological Evaluation of Medical Devices—Part 10: Tests for Irritation and Delayed-Typed Hypersensitivity. Two 25 mm×25 mm sections of the test article and control article were topically applied to the skin of each of three rabbits and left in place for 24 hours. The sites were graded for erythema and edema at 1, 24, 48 and 72 hours after removal of the single sample application.

Under the conditions of this study, very slight erythema and no edema were observed on the skin of the rabbits. The Primary Irritation Index for the test article was calculated to be 0.0. The response of the test article was categorized as negligible. The Goldshield™ treated face mask was considered very safe and caused virtually no skin irritation.

### Example 6

Sensitization Study: 2.5% Goldshield™ Antimicrobial Solution Treated Face Mask

A study was conducted on the guinea pig to evaluate the potential for delayed dermal contact sensitization of Goldshield 2.5% treated face mask, Batch: Test II Sep. 21, 2007. The study was conducted based on the requirements of the International Organization for Standardization 10993: Biological Evaluation of Medical Devices, Part 10: Tests for Irritation and Delayed-Type.

The test article (Goldshield 2.5%) treated face mask was occlusively patched for 6 to 8 hours to the intact skin of ten guinea pigs, three times a week, for a total of nine induction treatments over a three week period. The control article was similarly patched to five guinea pigs. Following a recovery period, the ten and five control animals received a challenge patch of the test article and the control article. All sites were observed for evidence of dermal reactions at 24 and 48 hours after patch removal.

Under the conditions of this study, the test article showed no evidence of causing delayed dermal contact sensitization in the guinea pig.

### Example 7

Toxicity Study: 2.5% Goldshield™ Antimicrobial Solution Treated Face Mask

An in vitro biocompatibility study, based on the requirements of the International Organization for Standardization (ISO 10993-5), was conducted on the test article, Goldshield 2.5% treated face mask, Batch: Test II Sep. 21, 2007, to determine the potential for cytotoxicity. Triplicate wells were dosed with a 1 cm×1 cm portion of the test article. Triplicate wells were dosed with a 1 cm length of high density polyethylene as a negative control. Triplicate wells were dosed with a 1 cm×1 cm portion of latex, as a positive control. Each was placed on an agarose surface directly overlaying a confluent monolayer of L-929 mouse fibroblast cells. After incubating at 37° C. in 5% CO₂ for 24 hours, the cell culture was examined macroscopically for cell decolorization around the test article and controls to determine the zone of cell lysis (if any). The culture was then examined microscopically (100×) to verify any decolorized zones and to determine cell morphology in proximity to the articles.

Under the conditions of this study, the test article showed evidence of causing slight cell lysis or toxicity. The test article met the requirements of the ISO since the grade was less than a grade 2 (mild reactivity). The negative control and the positive control performed as anticipated. The Goldshield™ treated face mask was considered very safe and caused virtually no cell toxicity.

It will be apparent to those skilled in the art that various modifications and variations can be made to various embodiments described herein without departing from the spirit or scope of the teachings herein. Thus, it is intended that various embodiments cover other modifications and variations of various embodiments within the scope of the present teachings.

What is claimed is:

1. A method of antimicrobially treating a substrate, comprising contacting the substrate with an effective amount of a water stable composition, comprising i) water; ii) a product that is formed by reacting in water: a) an organosilane of the formula Rₙ SiX₄₋ₙ where n is an integer of from 0 to 3; each R is, independently, a nonhydrolyzable organic group; and each X is, independently, a hydrolyzable group, with b) a polyol containing at least three hydroxy groups, wherein all of the hydroxy groups are separated by at least three intervening atoms, wherein the polyol is not hydroxyethyl cellulose, wherein the product is a monomer, oligomer, or a combination thereof, and iii) a wetting agent in an amount of 0.01% w/w or w/v to 10% w/w or w/v based on the weight of the total composition for a period of time sufficient to antimicrobially treat the substrate, wherein the substrate is a face mask or a dental product.

2. A method according to claim 1, wherein the substrate is a face mask.

3. A method according to claim 1, further comprising drying the antimicrobially treated substrate.

4. A method according to claim 1, wherein the organosilane comprises 3-(trimethoxysilyl)propyldimethyloctadecyl ammonium chloride, 3-(trimethoxysilyl)propylmethyldi(decyl)ammonium chloride, 3-chloropropyltrimethylsilane, 3-chloropropyltrimethoxysilane, octadecyltrimethoxysilane, or perfluorooctyltriethoxysilane and the polyol is pentaerythritol, dipentaerythritol, tripentaerythritol, tetrapentaerythritol, tris(hydroxymethyl)ethane, tris(hydroxymethyl)propane, tris(hydroxymethyl)nitromethane, tris(hydroxymethyl)aminomethane, or tris(hydroxymethyl)methanetrimethyl ammonium iodide.

5. A method according to claim 1, wherein the polyol comprises pentaerythritol, dipentaerythritol, tripentaerythritol, tetrapentaerythritol, tris(hydroxymethyl)ethane, tris(hydroxymethyl)propane, tris(hydroxymethyl)nitromethane, tris(hydroxymethyl )aminomethane, tris(hydroxymethyl)methane trimethyl ammonium iodide, or tetrakis(hydroxymethyl)phosphonium chloride.

6. A method according to claim 1, wherein the wetting agent comprises a non-ionic wetting agent.

7. A method according to claim 1, wherein the wetting agent comprises alcohol, ethoxylated nonylphenol, nonylphenol mono- and diethoylates, nonylphenoxy carboxylic acids, ethoxylated alcohol, glycol ethers, or a combination thereof.

8. A method according to claim 1, wherein the organosilane comprises 3-(trihydroxysilyl)propyldimethyloctadecyl ammonium chloride, the polyol comprises pentaerythritol, and the wetting agent comprises ethoxylated alcohol and glycol ethers.

9. A method of antimicrobially coating a substrate, comprising contacting the substrate with an effective amount of a water stable composition, comprising i) water; ii) a product that is formed by reacting in water: a) an organosilane of the formula Rₙ SiX₄₋ₙ where n is an integer of from 0 to 3; each R is, independently, a nonhydrolyzable organic group; and each X is, independently, a hydrolyzable group, with b) a polyol containing at least three hydroxy groups, wherein all of the hydroxy groups are separated by at least three intervening atoms, wherein the polyol is not hydroxyethyl cellulose, wherein the product is a monomer, oligomer, or a combination thereof, and iii) a wetting agent in an amount of 0.01% w/w or w/v to 10% w/w or w/v based on the weight of the total composition for a period of time sufficient to antimicrobially coat the substrate, wherein the substrate is a face mask or a dental product.

10. A method according to claim 9, wherein the substrate is a face mask.

11. A method according to claim 9, further comprising drying the antimicrobially treated substrate.

12. A method according to claim 9 wherein the organosilane comprises 3-(trimethoxysilyl)propyldimethyloctadecyl ammonium chloride, 3-(trimethoxysilyl)propylmethyldi(decyl)ammonium chloride, 3-chloropropyltrimethylsilane, 3-chloropropyltrimethoxysilane, octadecyltrimethoxysilane, or perfluorooctyltriethoxysilane and the polyol is pentaerythritol, dipentaerythritol, tripentaerythritol, tetrapentaerythritol, tris(hydroxymethyl)ethane, tris(hydroxymethyl)propane, tris(hydroxymethyl)nitromethane, tris(hydroxymethyl)aminomethane, or tris(hydroxymethyl)methanetrimethyl ammonium iodide.

13. A method according to claim 9 wherein the polyol comprises pentaerythritol, dipentaerythritol, tripentaerythritol, tetrapentaerythritol, tris(hydroxymethyl)ethane, tris(hydroxymethyl)propane, tris(hydroxymethyl)nitromethane, tris(hydroxymethyl)aminomethane, tris(hydroxymethyl)methane trimethyl ammonium iodide, or tetrakis(hydroxymethyl)phosphonium chloride.

14. A method according to claim 9, wherein the wetting agent comprises a non-ionic wetting agent.

15. A method according to claim 9, wherein the organosilane comprises 3-(trihydroxysilyl)propyldimethyloctadecyl ammonium chloride, the polyol comprises pentaerythritol, and the wetting agent comprises ethoxylated alcohol and glycol ethers.

16. A method according to claim 9, wherein the product is monomeric.

17. A method according to claim 9, wherein the product is oligomeric.

18. A method of antimicrobially enhancing a substrate, comprising contacting the substrate with an effective amount of a water stable composition, comprising i) water; ii) a product that is formed by reacting in water: a) an organosilane of the formula Rₙ SiX₄₋ₙ where n is an integer of from 0 to 3; each R is, independently, a nonhydrolyzable organic group; and each X is, independently, a hydrolyzable group, with b) a polyol containing at least three hydroxy groups, wherein all of the hydroxy groups are separated by at least three intervening atoms, wherein the polyol is not hydroxyethyl cellulose, wherein the product is a monomer, oligomer, or a combination thereof, and iii) a wetting agent in an amount of 0.01% w/w or w/v to 10% w/w or w/v based on the weight of the total composition for a period of time sufficient to antimicrobially enhance the substrate, wherein the substrate is a face mask or a dental product.

19. A method according to claim 18, wherein the organosilane comprises 3-(trihydroxysilyl)propyldimethyloctadecyl ammonium chloride, the polyol comprises pentaerythritol, and the wetting agent comprises ethoxylated alcohol and glycol ethers.

20. A method according to claim 18, wherein the effective amount kills at least 99.9% of microorganisms.

21. A method according to claim 18, wherein the effective amount kills 100% of microorganisms.
